(19) United States
(12) Patent Application Publication
(10) Pub. No.: US 2019/0336339 A1
(43) Pub. Date: Nov. 7, 2019

(54) PARANASAL SINUS ACCESS IMPLANT DEVICES AND RELATED PRODUCTS AND METHODS

(71) Applicant: SINOPSYS SURGICAL, INC., Boulder, CO (US)

(72) Inventors: Michael Lawrence Reo, Redwood City, CA (US); Brian James Willoughby, Denver, CO (US); Christopher Lee Oliver, Denver, CO (US); Harry Ross, Boulder, CO (US); Donald F. Schomer, Bellaire, TX (US); Monica Kellogg, Kiowa, CO (US)

(21) Appl. No.: 16/071,440

(22) PCT Filed: Jan. 27, 2017
(86) PCT No.: PCT/US2017/015447

§ 371 Date: Jul. 19, 2018

Related U.S. Application Data
(60) Provisional application No. 62/287,787, filed on Jan. 27, 2016.

(51) Int. Cl. A61F 9/007; B29D 23/00

(52) U.S. Cl. CPC A61F 9/00772; B29D 23/00; A61F 2250/0039; A61F 2250/0098; A61F 2240/001; A61F 2250/0067; A61F 2220/0008

(57) ABSTRACT

A paranasal sinus access implant device includes a head to be disposed in a location in the lacrimal apparatus and a conduit extending into a paranasal sinus when the implant device is implanted to provide a fluid communication path between the lacrimal apparatus and the paranasal sinus through an internal passage through the implant device. The head has a perimeter transverse to a longitudinal axis of the implant device and a flanged portion of the head extending 360 degrees around the axis. A side of the flanged portion is disposed toward and engages tissue in the lacrimal apparatus when the implant device is implanted. The head includes a concave undercut portion on the distal side of the flanged portion, with the concave undercut extending 360 degrees around the axis within the perimeter of the head. A method of manufacture includes modifying a preliminary form, which may include roughening a smooth surface and/or enlarging a preliminary form for the head including a preliminary form for the concave undercut portion. of the head.

### CROSS-REFERENCE TO RELATED APPLICATIONS

This application claims the benefit of U.S. provisional application No. 62/287,787 filed Jan. 27, 2016, the entire contents of which are incorporated herein by reference. This application incorporates by reference the contents of each and every portion of each and every one of the following: U.S. provisional patent application No. 62/028,682 entitled “APPARATUSES, TOOLS, KITS AND METHODS RELATING TO PARANASAL SINUS ACCESS” filed Jul. 24, 2014; international application no. PCT/US2015/042089 entitled “APPARATUSES, TOOL, KITS AND METHODS RELATING TO PARANASAL SINUS ACCESS” filed Jul. 24, 2015 and international application no. PCT/US2015/042099 entitled “PARANASAL SINUS ACCESS IMPLANT DEVICES AND RELATED PRODUCTS AND METHODS” filed Jul. 24, 2015.

### FIELD OF THE INVENTION

The invention relates to treatment of conditions of the paranasal sinuses, including with respect to paranasal sinus access implant devices and products and methods including paranasal access implant devices.

### BACKGROUND OF THE INVENTION

In the United States alone, 35 million people a year are treated for sinus infections, or sinusitis, and 7 million of those will suffer from chronic sinusitis and will have minimal response to prescription drug therapies. Conventional surgical interventions may be expected to, at best, offer only moderate symptomatic improvement but no cure.

Current drug therapies include oral administration as pills and nasal topical administration, neither of which is conducive to delivering adequate concentration of medication to the involved paranasal sinus. In addition to medication, frequent sinus irrigation can be helpful in flushing out debris and irritants, promoting ciliary function and obstructing viscous fluids, but patients are generally not able to adequately perform this procedure at home.

For patients with particularly severe symptoms, surgical drainage has been an option of last resort. An early surgical procedure was the Caldwell-Luc procedure, which involves creating a permanent fistula from the base of the maxillary sinus into the oral cavity by way of an incision into the canine fossa above the front upper canine teeth. More recently, other surgical access points to the paranasal sinuses have been attempted. A variety of endoscopic techniques have been developed that access the paranasal sinuses through the nose, including functional endoscopic sinus surgery (FESS) and balloon sinuplasty. All attempt to increase drainage or promote irrigation, but utilize different routes or tools. Implantation of an implant device through a surgically-formed fistula between the lacrimal apparatus and a paranasal sinus has been identified as a technique to provide direct access to the paranasal sinus, and through which a variety of medical treatments and medical procedures may be directed to the paranasal sinus. Though these surgical approaches are widely recognized, millions of patients continue to suffer long-term disability and discomfort. There continues to be a need for effective and convenient techniques to administer drugs directed to treatment of conditions of the paranasal sinuses.

### SUMMARY OF INVENTION

Paranasal sinus access implant devices may be configured to be implanted in a human to provide fluid access to a paranasal sinus through an internal passage of such a paranasal sinus access implant device, with the internal passage being accessible through an opening in a head of the paranasal sinus access implant device. The head may be configured to be disposed in the lacrimal apparatus in the orbit, for example between the medial canthus and the medial side of the adjacent eyeball, when the paranasal sinus access implant device is implanted to provide fluid access to a paranasal sinus. Such paranasal sinus access implant devices have significant potential for performance of medical procedures and treatments of paranasal sinuses, but such potential has not yet been realized. Treatment compositions may be delivered to a paranasal sinus through such an implant device by administering eye drops that may then flow through the implant device to a paranasal sinus. Fluid administrations may also be made by inserting a needle through the implant device to inject fluid directly into the paranasal sinus and medical procedures may involve passing a medical device through the implant device and into the paranasal sinus. However, use of such implant devices face challenges, including in relation to ease of implantation and control over the implantation procedure, patient comfort in relation to implanted devices and susceptibility of exposed surfaces of implant device to formation of biofilms following implantation. Also, mechanical interactions with the implant device following implantation, for example to perform a medical procedure through the implant device, have potential to structurally damage the implant device or to dislodge the implant device or disrupt the anchoring of the implant device in surrounding tissue. There are continued needs for implant devices and related procedures to address these challenges. Various aspects of this disclosure relate to implant device designs, methods and products that may address to some degree one or more of such needs.

A first aspect of the disclosure involves a paranasal sinus access implant device useful for implantation in a human to fluidly connect a lacrimal apparatus to a paranasal sinus through a fistula formed between the lacrimal apparatus and the paranasal sinus. Such a paranasal sinus access implant device may comprise:
a proximal end at a first longitudinal end of the implant device to be disposed in the lacrimal apparatus when the implant device is implanted and a distal end at a second longitudinal end of the implant device to be disposed in the paranasal sinus when the implant device is implanted;
a length longitudinally along the implant device between the proximal end and the distal end in a range of from 8 millimeters to 50 millimeters.
a conduit, located between the proximal end and distal end, to be disposed through a fistula between the lacrimal apparatus and the paranasal sinus when the implant device is implanted;
a head adjacent the proximal end and connected with the conduit, the head including a flanged portion to be disposed in the lacrimal apparatus when the implant device is implanted;
an internal passage through the head and the conduit, to provide a fluid communication path between the lacrimal apparatus and the paranasal sinus when the implant device is implanted, the internal passage having a width through at least a portion of the conduit in a range of from 0.5 millimeter to 2.5 millimeters; and further comprising any one or combination of more than one of the following features:
(a) an exposed surface of exposed material and having an average roughness (Ra) of not larger than 50 nanometers, wherein the exposed surface is exposed in the internal passage;
(b) an exposed surface having an average roughness of not larger than 50 nanometers, wherein the exposed surface is a first exposed surface and the average roughness is a first average roughness, and the implant device includes a second exposed surface having a second average roughness of at least 100 nanometers;
(c) an exposed surface of exposed material and having an average roughness of not larger than 50 nanometers, wherein the exposed surface extends over essentially all exterior of the implant device;
(d) the head has a perimeter transverse to a longitudinal axis of the implant device and the flanged portion of the head includes a concave undercut portion on a distal side of the flanged portion, with the concave undercut extending 360 degrees around the axis within the perimeter of the head;
(e) the internal passage includes a proximal port transition portion including a tapering cross-section that tapers in a direction from the proximal end toward the distal end of the implant device; and
(f) the internal passage includes a distal port transition portion with tapering cross-section that tapers in a direction from the distal end toward the proximal end of the implant device.

Another aspect of the disclosure involves a method for making a paranasal access implant device constructed of polymeric material, wherein the paranasal access implant device comprises:

a proximal end at a first longitudinal end of the device to be disposed in the lacrimal apparatus when the device is implanted and a distal end at a second longitudinal end of the device to be disposed in the paranasal sinus when the device is implanted;

a length longitudinally along the implant device between the proximal end and the distal end in a range of from 8 millimeters to 50 millimeters;

a conduit, located between the proximal end and distal end, to be disposed through a fistula between the lacrimal apparatus and the paranasal sinus when the device is implanted, the conduit comprising a distal portion to be disposed in the paranasal sinus when the implant device is implanted;

a head adjacent the proximal end and connected with the conduit, the head including a flanged portion to be disposed in the lacrimal apparatus when the device is implanted;

an internal passage through the head and the conduit, to provide a fluid communication path between the lacrimal apparatus and the paranasal sinus when the device is implanted, the internal passage having a width through at least a portion of the conduit in a range of from 0.5 millimeter to 2.5 millimeters;

the method comprising:

preparing a preliminary form of the implant device including preliminary forms of the head, conduit and internal passage; and

modifying the preliminary form, wherein the modifying comprises treating an exposed surface on an exterior of the preliminary form to prepare a roughened exposed surface having a higher average roughness than the exposed surface prior to the treating.

Another aspect of the disclosure involves a method for making a paranasal access implant device, wherein the paranasal implant device comprises:

a proximal end at a first longitudinal end of the device to be disposed in the lacrimal apparatus when the device is implanted and a distal end at a second longitudinal end of the device to be disposed in the paranasal sinus when the device is implanted;

a length longitudinally along the implant device between the proximal end and the distal end in a range of from 8 millimeters to 50 millimeters;

a conduit, located between the proximal end and distal end, to be disposed through a fistula between the lacrimal apparatus and the paranasal sinus when the device is implanted, the conduit comprising a distal portion to be disposed in the paranasal sinus when the implant device is implanted;

a head adjacent the proximal end and connected with the conduit, the head including at least one flanged portion to be disposed in the lacrimal apparatus when the device is implanted, the flanged portion having a distal side to be disposed toward tissue adjacent the fistula when the implant device is implanted and a proximal side opposite the distal side;

an internal passage through the head and the conduit, to provide a fluid communication path between the lacrimal apparatus and the paranasal sinus when the device is implanted, the internal passage having a width through at least a portion of the conduit in a range of from 0.5 millimeter to 2.5 millimeters;

wherein:

the flanged portion of the head has a concave undercut portion on the distal side, and the method comprises modifying a preliminary form of the implant device, the preliminary form including preliminary forms for the head, conduit and internal passage, with the preliminary form of the head including a preliminary form of the concave undercut portion, and the modifying comprises enlarging the head by adding a layer of added material to the proximal side and the distal side of the head, wherein the layer of added material is at least twice as thick on the proximal side of the head as on the concave undercut portion of the distal side of the head.

Another aspect of the disclosure involves a method for performing a medical procedure in relation to a paranasal sinus and/or administering a treatment composition or performing a medical operation through an implanted implant device of the first-noted aspect directed to the paranasal sinus.

Another aspect of the disclosure involves a paranasal sinus access implant device of the first-noted aspect packaged in sterile packaging in contact with a sterile storage liquid. The product may include a paranasal sinus access implant device, sterile storage liquid and sterile packaging, wherein the implant device and the storage liquid are disposed within the sterile packaging. In another aspect of the disclosure, a method for implanting a paranasal sinus access implant device of the first-noted aspect to fluidly connect a lacrimal apparatus and a paranasal sinus may include removing such an implant device from the sterile packaging of such a product, and implanting the implant device with a proximal end disposed in the lacrimal apparatus and a distal end disposed in the paranasal sinus to fluidly connect the lacrimal apparatus and the paranasal sinus through an internal passage of the implant device.

A number of feature refinements and additional features are applicable to any one or more of these or other aspects of the disclosure. These feature refinements and additional features may be used individually or in any combination within the subject matter of any such aspects. As such, each of the following features may be, but are not required to be, used with any other feature or combination of features in relation to the same aspect or any other aspect of the disclosure.

For brevity, paranasal sinus access implant devices are referred to herein as simply implant devices.

Various feature refinements and additional features are applicable to the paranasal sinus access implant device.

The paranasal sinus access implant device may include one or more material or geometry features, or be associated with one or more material, to at least partially address one of more of the needs noted above. The paranasal sinus access implant device may include at least one (any one, any combination of more than one or all) of the following features:
(i) the conduit comprises a first material having a first hardness and the head comprises a second material having a second hardness that is smaller than the first hardness;
(ii) the conduit comprises a distal portion to be disposed in the paranasal sinus when the implant device is implanted, the distal portion of the conduit comprising a structural portion of a first material having a first hardness and a skin portion supported by the structural portion, the skin portion including a second material having a second hardness that is smaller than the first hardness;
(iii) the head comprises a structural portion of a first material having a first hardness and a skin portion supported by the structural portion, the skin portion including a second material having a second hardness that is smaller than the first hardness;
(iv) the head has an exposed surface of a second material having a hardness of not larger than Shore A 45 durometer;
(v) the head comprises an exposed surface of a second material, the exposed surface having an average roughness (Ra) of not larger than 200 nanometers;
(vi) the conduit comprises a distal portion to be disposed in the paranasal sinus when the implant device is implanted, the distal portion of the conduit comprising an exposed surface of a second material, the exposed surface having an average roughness (Ra) of not larger than 200 nanometers;
(vii) the head comprises an exposed surface of a second material comprising a wetting agent to impart hydrophilicity to the exposed surface;
(viii) the conduit comprises a distal portion to be disposed in the paranasal sinus when the implant device is implanted, the distal portion of the conduit comprising an exposed surface of a second material comprising a wetting agent to impart hydrophilicity to the exposed surface;
(ix) the head comprises an exposed surface of a second material comprising an antimicrobial agent;
(x) the conduit comprises a distal portion to be disposed in the paranasal sinus when the implant device is implanted, the distal portion of the conduit comprising an exposed surface of a second material comprising an antimicrobial agent;
(xi) the head comprises a distal side having a concave surface disposed toward the distal end of the implant device;
(xii) the internal passage has a surface of a second material comprising a lubricity agent;
(xiii) at least a portion of the conduit is of a radiopaque material;
(xiv) the internal passage has a surface geometry comprising rifling;
(xv) the conduit comprises a distal extension portion that is extendable and contractible to lengthen and shorten a longitudinal length of a distal portion of the conduit disposed in the paranasal sinus when the implant device is implanted; and
(xvi) the implant device is packaged in sterile packaging in contact with a storage liquid.

Several of these features (i)-(xvi) involve use of a “second material”, which is a term used for convenience of reference and description and does not indicate a distinction from another material that may be used in the implant device, except as specifically identified as such. Reference to a “second material” is often to a material that may be selectively used to enhance a selected portion of the implant device, rather than as a main structural material of construction for the implant device or a portion of the implant device, although reference to a “second material” may also be to a main structural component of construction. Such a main structural material may sometimes be referred to herein for convenience as a “first material”. An implant device may include more than one such “second material” and may include one or more than one such “first material”. A material (e.g., “first material” or “second material”) that is exposed at a surface of an implant device may be referred to interchangeably as an “exposed material”. As will be appreciated, an implant device including any of the features (i)-(xvi) that recite both a “first material” and a “second material” (e.g., features (i)-(iii)) will include at least two different materials in the implant device, whereas an implant device including any of the features (i)-(xvi) that recite only a “second material” and not a “first material” (e.g., features (iv)-(x)) may be made entirely of such a “second material” or may include one or more other materials in addition to the recited “second material”.

Another aspect of the disclosure involves a method of making a paranasal sinus access implant device of the first aspect that includes at least one such second material. The method comprises: providing a preliminary form including a preliminary head structure and a preliminary conduit structure; and forming at least one said second material supported by one or both of the preliminary head structure and the preliminary conduit structure. The implant device may be according to the first aspect and may include at least one second material, for example as noted in any one of features (i)-(x) identified above applicable to the first aspect. A resulting structure may be used as a final paranasal sinus access implant device product or may be used as anew preliminary form for further processing in preparation of a final implant device product.

In relation to any of features (i)-(iii), in some implementations the first material may often have a hardness of at least Shore A 50 durometer, at least Shore A 55 durometer, at least Shore A 60 durometer, at least Shore A 65 durometer, at least Shore A 70 durometer or at least Shore A 75 durometer. Such a first material may often have a hardness not greater than Shore A 100 durometer, not greater than Shore A 95 durometer, not greater than Shore A 90 durometer, not greater than Shore A 85 durometer or not greater than Shore A 80 durometer. Such a first material may comprise, for example, silicone materials, polyurethane materials, silicone-urethane copolymers, silicone-polycarbonate copolymers, polycarbonate-urethane copolymers and silicone-polycarbonate-urethane copolymers. Such first material may be a structural material of construction for the implant device or a portion thereof. One preferred silicone material for many implementations includes polydimethylsiloxane as a primary silicone polymer component. In some preferred implementations, the first material has a hardness that is larger than the hardness of the second material by at least 10, at least 15, at least 20, at least 25 or at least 30 Shore A durometer units.

In relation to any one of features (i)-(x), a second material may in some implementations have a hardness that is not larger than Shore A 45 durometer, not larger than Shore A 40 durometer, not larger than Shore A 35 durometer, not larger than Shore A 30 durometer, not larger than Shore A 25 durometer or not larger than Shore A 20 durometer. In some implementations a second material may have a hardness of at least Shore A 5 durometer or at least Shore A 10 durometer. One preferred material for use in a second material to form a softer layer over a harder structural material is a silicone-based material, which may be a silicone polymer material or a silicone hydrogel material. In relation to features (ii) and (iii), such a skin portion with a softer second material may be in a layer having a thickness of at least 2 microns, at least 5 microns, or at least 10 microns, or at least 20 microns, and often having a thickness of not larger than 200 microns, not larger than 100 microns, not larger than 50 microns, not larger than 25 microns, not larger than 20 microns or not larger than 15 microns. Such a skin portion may have an exposed surface on the implant device (e.g., on the outside of the head or conduit or on walls of the internal passage), or may be covered by a further layer of material, for example of a wetting agent or antimicrobial agent. Silicone hydrogel materials may be silicone hydrogels such as are used in contact lenses. Such silicone hydrogels may include hydrophilic functionality to counteract hydrophobicity of polysiloxanes. Such hydrophilic functionality may be provided for example, by one or more materials such as pyrrolidone-based functionality (e.g., incorporation of N-vinylpyrrolidone or polyvinylpyrrolidone (PVP), also referred to as poly-N-vinylpyrrolidone or as poly-N-vinyl-2-pyrrolidone, acrylamide-based functionality (e.g. dimethyl acrylamide functionality), glycol-based functionality (e.g., polyethylene glycol) and/or TRIS-based functionality. Some example commercial silicone hydrogel material products include balafilcon A (Bausch & Lomb), Iotrafilcon A (CIBA Vision), Iotrafilcon B (CIBA Vision), comfilcon A (CooperVision), senofilcon A (Johnson & Johnson Vision Care) and galyfilcon A (Johnson & Johnson Vision Care). In relation to any one of features (iv)-(x), the recited second material may in some implementation have properties as described above for the first material of features (i)-(iii).

In relation to either one of features (v) or (vi), such an exposed surface may have an average roughness (Ra) of not larger than 200 nanometers, not larger than 100 nanometers, not larger than 50 nanometers, not larger than 35 nanometers, not larger than 25 nanometers, not larger than 20 nanometers, not larger than 15 nanometers or not larger than 10 nanometers. Such an average roughness (Ra) may often be at least 1 nanometer, or at least 2 nanometers or at least 5 nanometers. Average roughness Ra may be determined by any suitable analytical technique. Average roughness (Ra) may be as determined by optical non-contact profilometry, laser profilometry or atomic force microscopy (AFM). In preferred implementations, the Average roughness (Ra) is as determined by optical non-contact profilometry. Also, the location of such an exposed surface with such an average roughness (Ra) may be located on any exposed surface of an implant device, in addition to or as an alternative to being located on the head or distal portion of the conduit. Such an exposed surface may be an exterior surface, such as an exterior exposed surface of the head or conduit, or may be an exposed internal surface, such as a surface that is exposed in the internal passage. Such an exposed surface with such an average roughness (Ra) may extend over all or essentially all of the exterior of the implant device. In some contemplated implementations, a first exposed surface on a first portion of an implant device (e.g., on a portion of the head or the conduit) may have a first average surface roughness that is small, for example not larger than 50 nanometers, and a second exposed surface on a second portion of the implant device (e.g., on a different portion of head or the conduit) that has a larger second average surface roughness (Ra), for example at least 100 nanometers.

In relation to either one of features (vii) or (viii), such a wetting agent may be any polymeric or non-polymeric material that imparts increased hydrophilicity to the exposed surface relative to material of the implant device not including such a wetting agent. Such a wetting agent may be immobile (e.g., through cross-linking or polymer functionalization) or may elute over time (e.g., deposited surface coating or leachable component mixed into material composition). The wetting agent may be or include one or more surfactants. Multiple wetting agents may be used together. A wetting agent may include multiple components that together provide desired wettability. Some example wetting agents include polyvinylpyrrolidone (PVP), polyethylene glycol (PEG), polyethylene oxide, polypropylene oxide, poly(oxyethylene)-poly(oxybutylene) copolymers, hydroxypropylmethylcelluluse (HPMC), polyvinyl alcohol (PVA), poloxamines and hyaluronic acid. The wetting agent may be applied as a coating, alone or mixed with another material, may be part of a material of construction, or may be bonded to the surface of the implant device. For example, polyethylene glycol may be grafted onto silicone elastomers, such as by atmospheric pressure plasma induced grafting. As another example, polyethylene glycol may be copolymerized with some silicone materials (e.g., with polydimethyl siloxane). As other examples, any of these example materials could be deposited on a surface as a coating. A wetting agent may be a part of a silicone hydrogel composition at the exposed surface.

In relation to either one of features (ix) or (x), such an antimicrobial agent may be any material that has antimicrobial properties (e.g., kills or inhibits growth of or interaction with microorganisms), which may significantly help to prevent formation of biofilms on such exposed surfaces having the antimicrobial agent. Such an antimicrobial agent may, for example, be of a type as have been described for use with catheters and/or contact lenses. Such an antimicrobial agent may be immobile (e.g., through covalent or strong ionic attachment) or may elute over time (e.g., deposited surface coating or leachable component mixed into material composition). Multiple antimicrobial agents may be used together. An antimicrobial agent may include multiple components that together provide antimicrobial activity. An antimicrobial agent may be incorporated into or applied to a polymer composition of the implant device that is exposed at an exterior surface of the implant device and/or that is exposed at a surface of the walls of the internal passage. An antimicrobial may be incorporated into a material prior to using the material to fabricate an implant device or a preliminary form for an implant device, or may be incorporated into the material following formation of a preliminary form. An antimicrobial agent may be intermixed with other components, may be in the form of an adhered coating or may be covalently or otherwise attached to a polymeric material (e.g., polysiloxane) or other material of the implant device. Some example antimicrobial agents include silver (including for example in the form of silver metal or silver salts and silver oxides), poly(ethylene oxide) (PEO), PEG and antimicrobial peptides. In some implementations, antimicrobial peptides are preferred. Some other example antimicrobial agents include chlorhexidine and/or silver sulfadiazine impregnation, minocycline-rifampicin impregnation, silver-containing nanoparticles impregnation, antimicrobial peptide impregnation or ionic or covalent incorporation (e.g., small cationic peptides, such as for example beta defensins, indolicidin, cecropin A, and magainins, melinines, protattins and lactoferrins). Some other examples of antimicrobial agents and methods and agents of attachment or adhesion may be found, for example, in the following publications, the entire contents of which are incorporated herein by reference:
Sousa, Cláudia et al., Mini-Review: Antimicrobial central venous catheters—recent advances and strategies, Biofouling, Vol. 27, No. 6, 609-620, July 2011.
Danese, Paul N, Antibiofilm Approaches: Prevention of Catheter Colonization, Chemistry & Biology, Vol. 9, 873-880, August 2002.
Gu, Xiaobo et al., Optimization of Ceragenins for Prevention of Bacterial Colonization of Hydrogel Contact lenses, IOVS, Vol. 54, No. 9, 6217-6223, September 2013, The Association for Research in Vision and Ophthalmology, Inc., www.iovs.org.
Sambhy, Vaun et al., Silver Bromide Nanoparticle/Polymer Composites: Dual Action Tunable Antimicrobial Materials, J. Am. Chem. Soc., Vol. 128, No. 30, 9798-9808, 2006.
Li X, et al., Antimicrobial functionalization of silicone surfaces with engineered short peptides having broad spectrum antimicrobial and salt-resistant properties, Acta Biomaterialia, Vol. 10, Issue 1, 258-266, January 2014.
Mishra, Biwajit et al., Site specific immobilization of a potent antimicrobial peptide onto silicone catheters: evaluation against urinary tract infection pathogens, J. Mater. Chem. B, Vol. 2, 1706-1716, 2014.
Cole, Nerida et al., In Vivo Performance of Melimine as an Antimicrobial Coating for Contact Lenses in Models of CLARE and CLPU, IOVS, Vol. 51, No. 1, 390-395, January 2010.
Balaban, Naomi et al., Prevention of Staphylococcus aureus biofilm on dialysis catheters and adherence to human cells, Kidney International, Vol. 63, 340-345, 2003.
U.S. Pat. No. 7,282,214.
U.S. Patent Application Publication 2002/0068013.

Antimicrobial peptides may be deposited on a surface by themselves, or in a mixture with another material (e.g., polymeric material) that helps to immobilize the antimicrobial peptide at the exposed surface. For example, such an antimicrobial agent may be mixed with a polymeric material that helps to immobilize the antimicrobial agent. Such a polymeric material may be a wetting agent, for example any of the polymeric wetting agents listed previously. In some preferred alternatives, an antimicrobial peptide may be covalently attached to a polymeric material, for example to silicone materials, according to known methods. Such antimicrobial peptides may be bonded directly to exposed material of a preliminary implant device form or may be pre-bonded to a polymer that is then deposited over surfaces of such a preliminary implant device form. Such covalent bonding, or attachment, may be through the use of one or more coupling agents. Coupling agents may be used to provide a stable bond improving affinity and adhesion between dissimilar materials (e.g., between a polymeric material of the implant device and an antimicrobial peptide or other antimicrobial agent). Coupling agents may be organic, inorganic and organic-inorganic. Some example organic coupling agents include isocyanates, anhydrides, amides, imides, acrylates, chlorotriazines, epoxides and organic acids, and various monomers, polymers and copolymers. Some example inorganic coupling agents include silicates. Some example organic-inorganic coupling agents include silanes and titanates. Silanes are one preferred group of coupling agents for covalent bonding to silicone and silicone hydrogel materials. In the case of polyethylene oxide as an antimicrobial agent, the polymer may be deposited over an exposed surface of a preliminary implant device form, or may be covalently attached to silicone or silicone hydrogel surfaces.

When an implant device includes an exposed surface of a second material, such as in any of features (iv)-(x), such an exposed surface may be in the form of a continuous or discontinuous surface feature. By a continuous surface feature it is meant a contiguous surface area of homogeneous surface properties. By a discontinuous surface feature it is meant a surface area that includes multiple distinct surface areas with different surface properties, such as distinct domains of a surface property (e.g., surface spots of wetting agent, antimicrobial agent, etc.) separated by surface areas having a different surface property (e.g., not including the wetting agent, antimicrobial agent, etc. that is present in the spots). The exposed surface, whether continuous or discontinuous, may have an area of at least 1 square millimeter, at least 2 square millimeters, at least 3 square millimeters, at least 5 square millimeters, at least 10 square millimeters, at least 20 square millimeters, at least 50 square millimeters or at least 100 square millimeters. By area of a discontinuous exposed surface it is meant the entire area of exposed surface including all surface phases within the area perimeter to which the distinct domains (e.g., surface spots) extend. In some preferred implementations the exposed surface is in the form of a continuous surface feature. When an exposed surface of a second material is on an exterior distal portion of the conduit, such as in any of features (vi), (viii) or (x), the exposed surface may extend for at least 2 millimeters, at least 3 millimeters or at least 5 millimeters or at least 10 millimeters along a longitudinal length of the conduit or even over the entire length or essentially the entire length of the conduit.

An exposed surface of a second material on the head of an implant device may be disposed toward and/or away from tissue adjacent the fistula (e.g., toward or away from conjunctiva in the orbit) when the implant device is implanted. An exposed surface of a second material on the head may extend over all or essentially all of the exterior of the head. An exposed surface of a second material on the conduit may extend entirely or essentially entirely around an exterior circumference of at least a portion of the conduit, and may extend over all or essentially all of the exterior of the conduit, including portions of the conduit disposed in the fistula when implanted. An exposed surface of a second material may extend over a portion, all or essentially all of the walls of the internal passage of the implant device.

In some implementations, a single component may provide multiple effects, or multiple components together may provide multiple effects. For example a single component, or multiple components together, may serve as both a wetting agent and an antimicrobial agent.

In relation to any one of features (i)-(iii), there may be a plurality of layers that grade from harder to softer toward the surface of the implant device. For example, one or more additional material layer may be disposed between the first material and the second material, wherein the intermediate material has a hardness intermediate between the hardness of the first material and the hardness of the second material. For example, a material may be disposed between the first material and the second material that has such an intermediate hardness that is at least 10 Shore A durometer units smaller than the hardness of the first material and at least 10 Shore A durometer units larger than the hardness of the second material. Such an intermediate material may have a hardness in a range having a lower limit of Shore A 10 durometer, Shore A 15 durometer, Shore A 20 durometer or Shore A 25 durometer and an upper limit of Shore A 50 durometer, Shore A 45 durometer, Shore A 40 durometer or Shore A 35 durometer. Such an intermediate material may in some preferred implementations be a silicone material.

In relation to feature (xii), the lubricity agent may be any material that provides enhanced lubricity to such a surface of the internal passage. Some example lubricity agents include flurosilicone materials, very smooth silicone films and poly(p-xylylene) polymers. Flurosilicone materials include siloxane-based polymers including one of more fluoro groups. One example flurosilicone that may be used as a lubricity agent is the copolymer dimethyl methyl trifluoropropylsiloxane. An example of a material for a very smooth silicone film providing lubricity is a film made from silane-based materials such as ethyltriacetoxysilane. Some example poly (p-xylylene) polymers for providing lubricity include Parylene N or Parylene C polymer products (Para Tech Coating, Inc.). A coating of or including such a lubricity material may be formed on walls of the internal passage by any suitable technique, such as deposition from a solution or slurry followed by drying and curing as needed. Poly(p-xylylene) polymers may be deposited, for example, by chemical vapor deposition. Such a coating of or containing a lubricity agent may have any desired thickness to provide the desired level of lubricity. Such a coating may often have a thickness in a range having a lower limit of 1 micron, 2 microns, 5 microns or 10 microns and an upper limit of 200 microns, 100 microns, 50 microns, 25 microns or 15 microns.

With respect to feature (xiii) the radiopaque material may be in the form of a radiopaque additive or the radiopaque material may comprise a polymeric material that may have mixed therein a radiopaque additive, for example a particulate filler having a high radiopacity, also referred to as radiodensity. Examples of some radiopaque additives that may be used as or to impart radiopacity to a material include one or more of barium sulfate, titanium metal, tantalum metal, gold metal, platinum metal, iodine, bismuth subcarbonate, bismuth trioxide, bismuth oxychloride and tungsten. Some other radiopaque additives may be in the form metal beads or metal wires (e.g., of any of the metals listed above) embedded in a polymer matrix material. Such radiopaque additives may be added into a resin composition as a solid particulate filler, or may be present in solution in an initial composition with the radiopaque material then precipitating during manufacture processing. The radiopaque material may have any desired amount of the radiopaque additive to provide a desired level of radiopacity to the material, which will depend in part on the radiodensity properties of the particular radiopaque additive. In many implementations, the radiopaque additive may be present in the radiopaque material in an amount in a range having a lower limit of 1 weight %, 5 weight %, 10 weight %, 15 weight % or 20 weight % and an upper limit of 90 weight %, 80 weight %, 70 weight %, 60 weight %, 50 weight % or 40 weight %. The radiopaque material may include one or more polymeric components in addition to the radiopaque additive. Such polymeric components may be any of the polymeric materials described herein for making any portion of the implant device. In some implementations, the polymeric material will include a silicone material. In various implementations, at least a portion of the conduit including the radiopaque material may have a radiodensity of at least 50 Hounsfield units, at least 100 Hounsfield units, at least 200 Hounsfield units, at least 300 Hounsfield units or at least 400 Hounsfield units. Such radiodensity may in various implementations often be not larger than 1000 Hounsfield units, not larger than 900 Hounsfield units, not larger than 800 Hounsfield units or not larger than 700 Hounsfield units. In some preferred implementations, a radiopaque portion of the conduit extends for at least 2 millimeters, at least 3 millimeters, at least 4 millimeters, at least 5 millimeters, or at least 6 millimeters along a longitudinal length of the conduit. In some implementations, the radiopaque portion of the conduit may include at least such a portion of the longitudinal length of the conduit that includes a distal end of the conduit. Such a radiopaque portion may have a proximal end toward the head that is at least 0.5 millimeter, at least 1 millimeter, at least 2 millimeters or at least 3 millimeters distal of the head. In some implementations, the entire conduit may be made of radiopaque material. In other implementations, the head of the implant device may also be made of radiopaque material. However, in some preferred implementations at least a portion of the head, and more preferably substantially all of the flanged portions of the head, and even more preferably substantially all of the head, has a radiodensity that is smaller than the radiodensity of the radiopaque portion of the conduit, and which may often be a radiodensity of no larger than 75 Hounsfield units, no larger than 60 Hounsfield units, no larger than 40 Hounsfield units, no larger than 30 Hounsfield units or no larger than 20 Hounsfield units. In some particularly preferred implementations, the head does not contain a radiopaque additive. Having a radiopaque conduit portion permits easy radio imaging of that portion of the conduit during an implantation procedure to ensure proper positioning of the conduit for implantation and/or for long-term monitoring of the positioning of that portion of the conduit following implantation, for example to detect possible migration of the implant device post implantation. Having a substantially transparent or translucent head (preferably having both high radiotransparency and high transparency to visible light) makes the implant device less visible (e.g., in the orbit) and therefore more aesthetically pleasing to patients when implanted. At least a portion of the head, and preferably at least flanged portions of the head, may be visible light translucent or even visible light transparent. At least a portion of the head, and preferably at least flanged portions of the head may have a refractive index of not larger than 1.5, not larger than 1.45, not larger than 1.4 or not larger than 1.35. The refractive index may often be at least 1.3.

With respect to feature (xiv), the rifling may include a spiraling groove, recess, or other surface geometry in a wall of the internal passage, and may extend down a portion or all of the longitudinal length of the internal passage.

With respect to feature (xv), the extension portion may include or be a pleated or accordion-like structure that extends and contracts.

With respect to feature (xvi), the sterile packaging, implant device and storage liquid may be, for example, as described for the packaged product aspect of the disclosure.

Various feature refinements and additional features are applicable to a method for making a paranasal sinus access implant device.

A method for making a paranasal sinus access implant device may include forming such a second material over at least a portion of a preliminary head structure, over at least a portion of a preliminary conduit structure or over at least a portion of a preliminary internal passage structure. The second material may be formed over an existing first material or a previously provided second material, so that an implant device structure may include multiple second materials with one second material disposed over a prior second material. Forming a second material may include modifying the properties of an existing material of a preliminary structure, such as by impregnating an existing material with an additive or reacting an additive at the surface of an existing material. Second materials formed during final processing to prepare a final implant product may be exposed at a surface of the implant device product to provide a particular property for the implant device, for example a very smooth surface, hydrophilicity and/or antimicrobial activity. In some implementations, providing a preliminary form for use in the method may include modifying a prior preliminary form. In some implementations, providing a preliminary form may include molding a polymeric composition in the shape of the preliminary form, which may include curing a resin composition. Such molding techniques may include injection molding, compression molding and transfer molding. Such molding may be performed on an existing structure, such as an extruded tubular shaft forming a base structure for an implant device. Other features of an implant device (e.g., head and anchor protrusions) may be molded around the extruded form. The providing a preliminary form on which a second material may be formed may include removing flash from a molded article (mold flash) to provide a smoother surface on which to apply a second material. Forming a second material may include any deposition or impregnation technique. Some example techniques include dip molding or spray molding to apply a thin layer of a second material. Such dip molding or spray molding may include applying a precursor solution with at least one precursor for the second material, drying the precursor solution to leave residual precursor on the preliminary form and optionally curing as necessary residual precursor to form a final polymeric composition of the second material. In some implementations, a polymeric composition of the second material may include a thermoset polymer, and the curing may include heating precursor on the preliminary form to cure the thermoset composition. During drying, the implant device form may be subjected to three dimensional spinning to promote even distribution of deposited material on surfaces of the preliminary implant device on which the material is deposited. The forming a second material may include first forming an intermittent material over a desired portion of an implant device structure and then forming a second material over at least a portion of the preliminary material. The preliminary material may also be a second material, which may be the same or different than the subsequently applied second material. Forming a second material may include forming a preliminary material over a desired portion of the preliminary implant device form and then modifying the preliminary material to form the composition of the desired second material. For example, such modifying may include adding an additive material (e.g., antimicrobial agent, wetting agent, lubricity agent) to the preliminary material.

Various other feature refinements and additional features are applicable to the paranasal sinus access device of the first aspect.

The implant device may be configured to be implanted between the lacrimal apparatus in the orbit and a paranasal sinus (e.g., ethmoid sinus, frontal sinus or maxillary sinus), wherein when so implanted the proximal end is disposed in the lacrimal apparatus within the orbit and the distal end is disposed in the paranasal sinus.

The conduit may be configured so that an exterior of the conduit comprises an anchoring surface feature that assists to anchor the implant device when the implant device is implanted. The anchoring surface feature includes protrusion areas and recess areas. The second minimum wall thickness may occur at a location corresponding with at least one of the recess areas. The implant device may be configured so that when implanted the conduit is disposed through the fistula with at least a portion of the recess areas disposed within the fistula and with at least a portion of the protrusion areas disposed in the fistula and engaging tissue exposed within the fistula to anchor the implant device. The structural and mechanical characteristics of protrusion occurrences in the protrusion areas may affect anchoring performance of the protrusion areas. The height of the protrusion areas relative to the recess areas may affect anchoring effectiveness when the implant device is implanted. A larger height may provide greater anchor effectiveness, but also may involve a larger overall width of the implant device that must be inserted into the fistula. The protrusion areas may have a height relative to the recess areas of at least 0.1 millimeter, at least 0.2 millimeter, at least 0.25 millimeter, at least 0.3 millimeter or at least 0.35 millimeter. The protrusions areas may have a height relative to the recess areas of no greater than 2 millimeters, no greater than 1.5 millimeter, no greater than 1 millimeter, no greater than 0.75 millimeter, no greater than 0.5 millimeter, no greater than 0.45 millimeter or no greater than 0.4 millimeter. The height may be of particular protrusion occurrences relative to adjacent areas of recesses. Protrusion occurrences are also referred to herein as anchor protrusions. Such anchor protrusions may be configured to flexibly deform when the conduit is inserted through the fistula for implantation, for example to flexibly deform in a direction opposite the direction of insertion when the anchor protrusions contact tissue disposed in the fistula during insertion. After insertion, the anchor protrusions may over time return to their original shape and extend deeper into adjacent tissue to better anchor the implant device. The mechanical properties of the anchor protrusions may be influenced by materials of construction. Preferred materials of construction for the protrusion areas, and also for the other structured portions of the implant device, are polymeric materials. The polymeric materials may preferably be medical grade materials. Some preferred polymeric materials are silicones and polyurethanes. For enhanced performance, a structural material of construction should have a rigidity that interacts positively with tissue in the vicinity of the fistula, for example to promote load sharing and good anchoring. One preferred material of construction for structural purposes (e.g., for use or a “first material” or a structural “second material”) is a polymeric material (e.g. silicone, polyurethane, silicone-urethane copolymers, silicone-polycarbonate copolymers, polycarbonate-urethane copolymers or silicone-polycarbonate-urethane copolymers) having a durometer (Shore A) in a range having a lower limit of 50, 60, 70 or 80 and an upper limit of 100, 80, 70 or 60, provided that the upper limit must be larger than the lower limit. One preferred range is for a durometer (Shore A) of 60-100, with a range of 70-100 or 80-100 being even more preferred. For some implementations the polymeric material has a durometer (Shore A) of about 60, of about 70, of about 80 or of about 100. Mechanical properties of the protrusion occurrences of the protrusion areas will also be affected by the geometry of the protrusion occurrences. The protrusion occurrences may have a width that tapers, or narrows, in a direction from a base toward a top of the protrusion occurrences, with the base being a portion of a protrusion occurrence disposed toward the internal passage of the conduit and a top of the protrusion occurrence being the extremity of the protrusion occurrence away from the internal passage of the conduit. The width may be transverse to the length of the conduit. The protrusion occurrences may have a width at the base that is no larger than 2 millimeters, no larger than 1.5 millimeters, no larger than 1.25 millimeters, no larger than 1 millimeter or no larger than 0.75 millimeter. One or more of the protrusion occurrences may have a width at the base that is at least 0.2 millimeter, at least 0.3 millimeter, at least 0.5 millimeter, at least 0.75 millimeter or at least 1 millimeter. The protrusion occurrences may have a width adjacent the top that is no larger than 0.75 times width at the base, no larger than 0.5 times the width at the base, or no larger than 0.25 times the width at the base. The protrusion occurrences may have a width midway between the base and the top that is no larger than 0.8 times the width of the base, no larger than 0.7 times the width of the base, no larger than 0.6 times the width of the base or no larger than 0.5 times the width at the base.

The protrusion areas may be provided by a single protrusion occurrence feature located to correspond with the interior of the fistula when the implant device is implanted. In more preferred implementations, the protrusion areas include multiple protrusion occurrences spaced on the exterior of the conduit. The protrusion occurrences may have a center-to-center spacing, in one or more directions, of at least 0.5 millimeter, at least 0.75 millimeter, at least 1 millimeter, at least 1.25 millimeters, at least 1.4 millimeters or at least 1.75 millimeters. The protrusion occurrences may have a center-to-center spacing of no greater than 2.5 millimeters, no greater than 2 millimeters, no greater than 1.75 millimeters or no greater than 1.6 millimeters. The protrusion occurrences may have a center-to-center spacing longitudinally along the conduit. The protrusion occurrences may have a center-to-center spacing that is at least 0.5 times the base width of the protrusion occurrences, or at least 1 times the base width of the protrusion occurrences or at least 2 times the base width of the protrusion occurrences. The protrusion occurrences may have a center-to-center spacing that is no more than 5 times a base width of the protrusion occurrences, no more than 4 times a base width of the protrusion occurrences, no more than 3 times a base width of the protrusion occurrences or no more than 2 times a base width of the protrusion occurrences.

The protrusion areas may be located on a longitudinal portion of the conduit that includes at least a portion of the conduit that will be disposed within a fistula when the implant device is implanted. The protrusion areas may be on a longitudinal portion of the conduit that extends for at least 2 millimeters along the length of the implant device, that extends for at least 3 millimeters along the length of the implant device, that extends for at least 4 millimeters along the length of the implant device, that extends for at least 5 millimeters along the length of the implant device, that extends for at least 6 millimeters along the length of the implant device or that extends for at least 8 millimeters along the length of the implant device. A longitudinal portion of the conduit including the protrusion areas may be no longer than 30 millimeters, no longer than 25 millimeters, no longer than 20 millimeters, no longer than 15 millimeters or no longer than 10 millimeters. A longitudinal portion of the conduit including the protrusion areas may be disposed at least 2 millimeters from the proximal end of the implant device, at least 3 millimeters from the proximal end of the implant device, at least 4 millimeters from the proximal end of the implant device or at least 6 millimeters from the proximal end of the implant device. A longitudinal portion of the conduit including the protrusions may be disposed at least 1 millimeter, at least 2 millimeters, at least 3 millimeters, at least 4 millimeters or at least 6 millimeters from a head of the implant device. Providing significant distance between the head and commencement of the protrusion areas permits the head to better “float” on the surface of tissue, which may enhance patient comfort and device performance. The protrusion areas may be disposed along a longitudinal portion of the conduit with the protrusion areas covering no more than 40% of the area along the longitudinal portion of the conduit, 35% of the area along that longitudinal portion of the conduit, no more than 25% of the area along that longitudinal portion of the conduit or not more than 20% of the area along that longitudinal portion of the conduit. Providing significant spacing between protrusion occurrences may permit better engagement of tissue by the anchoring surface feature. Some or all of the protrusion occurrences may be on the second longitudinal portion of the conduit.

The protrusion areas may comprise at least one circumferential ridge. By circumferential ridge it is meant a ridge that extends around an entire circumference of the conduit. The protrusion areas may comprise at least two, at least three, at least five or at least six circumferential ridges. The protrusion areas may in some implementations comprise not more than 20, not more than 15 or not more than 10 circumferential ridges. The protrusion areas may comprise a spiral ridge. Such a spiral ridge may extend along a longitudinal portion of the conduit. The protrusion areas may comprise a knob or may comprise multiple knobs. The anchoring surface feature may comprise a textured surface, with the protrusion areas comprising protruding portions of the textured surface and the recess areas comprising recess portions of the textured surface.

The length of the implant device may be selected to provide sufficient conduit length for extending through the entire length of the fistula plus any extension distance desired in the lacrimal apparatus proximal to the fistula and in the paranasal sinus distal to the fistula. The length of the implant device and/or of the conduit may be in a range having a lower limit of 2 millimeters, 3 millimeters, 4 millimeters, 5 millimeters, 8 millimeters, 10 millimeters, 12 millimeters or 15 millimeters and an upper limit of 50 millimeters, 40 millimeters, 30 millimeters, 25 millimeters, 20 millimeters, 15 millimeters or 10 millimeters, provided that the upper limit is larger than the lower limit. One preferred range for some implementations when the fistula is between the orbit and the ethmoid sinus or the maxillary sinus is for the length of the implant device and/or for the length of the conduit to be in a range of from 10 millimeters to 30 millimeters, with a range of from 15 millimeters to 25 millimeters being more preferred. By length of the implant device or the conduit it is meant the dimension longitudinally along the implant device or the conduit, as the case may be, from the proximal end to the distal end of the implant device or the conduit, and may be along a longitudinal axis through the internal passage. The length may be a straight line, for example when the internal passage is straight, or the length may be curvilinear or some other shape, for example when the internal passage is not linear. When a reference is made herein to transverse to the length, the reference is to a right angle to the longitudinal direction of the length at that point (e.g., right angle to a line of the length or to a line tangent to a curve of the length). When the conduit includes an extension portion that is extendable and contractible to lengthen and shorten the conduit, the implant device that is fully shortened by full contraction and that is fully lengthened by full extension may be within the noted length ranges.

The implant device may advantageously be designed with a conduit of appropriate width dimensions to fit snuggly within a desired size of fistula. The implant device may have a first exterior width dimension defined by a maximum extent of the protrusion areas transverse to the length of the implant device, with the first exterior width being within a range having a lower limit of 0.75 millimeter, 1 millimeter, 1.25 millimeters, 1.5 millimeters, 1.75 millimeters or 2 millimeters and an upper limit of 8 millimeters, 7 millimeters, 6 millimeters, 5 millimeters, 4 millimeters, 3 millimeters, 2.5 millimeters, 2 millimeters or 1.75 millimeters, provided of course that the upper limit must be larger than the lower limit. The conduit may have a second width dimension defined by the minimum extent of the recess areas transverse to the length of the implant device, and which second exterior width dimension will be smaller than the first exterior width dimension defined by the protrusion areas. The second exterior width dimension defined by the recess areas may be smaller than the first exterior width dimension defined by the protrusion areas by an amount within a range having a lower limit of 0.2 millimeter, 0.25 millimeter, 0.35 millimeter, 0.5 millimeter, 0.6 millimeter or 0.7 millimeter and having an upper limit of 1.5 millimeters, 1 millimeter, 0.9 millimeter or 0.75 millimeter. The height of the protrusion areas may be one-half the difference between the first exterior width and the second exterior width. Either one of or each one of the first exterior width and the second exterior width may be the diameter of a circle.

The implant device may include one or a plurality of side openings (also referred to as side ports) through the conduit wall of a distal portion of the conduit, which distal portion may be or include a portion of the conduit that is designed to be disposed within a paranasal sinus when the implant device is implanted to provide fluid access through the implant device to the paranasal sinus. The side openings may be open into the internal passage through the conduit and may provide a passage for fluid communication between the internal passage of the implant device and the paranasal sinus even if the distal opening of the internal passage at the distal end of the conduit were to become blocked or restricted for some reason. One or more of the side openings may be through a wall of the second longitudinal portion of the conduit, and may be though a wall having the second minimum wall thickness. One or more of the side openings may be located within one or more recess areas of an anchoring surface feature of the conduit (e.g., between circumferential ridges). With a thinner minimum wall thickness in the second longitudinal portion of the conduit than the first longitudinal portion of the conduit, the second longitudinal portion of the conduit, and in particular near a distal end of the second longitudinal portion of the conduit, may be more prone to restriction due to collapse of the conduit at or near the distal end, and the side openings provide an alternative fluid access to the paranasal sinus.

The implant device may include a head adjacent to the conduit at the proximal end of the implant device. The implant device may be configured so that when the implant device is implanted to fluidly connect between a location in the lacrimal apparatus in the orbit and a paranasal sinus (e.g., frontal, ethmoid or maxillary sinus), the head is disposed in the lacrimal apparatus in the orbit, such as in the conjunctival cul-de-sac. The head may comprise a flanged tissue engagement surface on a side of the head disposed toward the conduit and configured to engage tissue outside of and adjacent to the fistula when the implant device is implanted. The flanged tissue engagement surface may be a flat surface. The flanged tissue engagement surface may have non-flat surface features configured to improve seating of the surface against tissue, such as for example to inhibit rotation of the implant device within the fistula after implantation. The head may have a face surface opposite the flanged tissue engagement surface and also disposed away from the conduit and disposed away from tissue engaged by the flanged tissue engagement surface when the implant device is implanted. The face surface may be substantially flat. The face surface may be disposed at the proximal end of the implant device and the internal passage may open at the face surface. The separation distance between the face surface and the flanged tissue engagement surface may be in a range having a lower limit of 0.25 millimeter, 0.5 millimeter or 0.75 millimeter and having an upper limit of 2 millimeters, 1.5 millimeters or 1 millimeter. Such separation distance need not be constant across the flanged tissue engagement surface and face surface. A maximum separation distance between the face surface and the flanged tissue engagement surface may be referred to as the depth of the head, and such depth may be in a range described above for the separation distance between the face surface and the flanged tissue engagement surface. The flanged tissue engagement surface need not be continuous and may be divided into multiple distinct surface portions. For example, the flanged tissue engagement surface may include a first flanged portion disposed to one side of the internal passage and a second flanged surface portion disposed to a second side of the internal passage that is opposite the first side. Each of the face surface and the flanged tissue engagement surface may have a length dimension that represents a maximum separation distance between points on an outer edge of the respective surface, and may each have a width dimension that is a maximum separation distance between points on the outer edge transverse to the length dimension. The length dimensions of the face surface and the flanged tissue engagement surface may be the same or may be different. The width dimensions of the face surface and the flanged tissue engagement surface may be the same or may be different. The face surface and the flanged tissue engagement surface may have corresponding outer edges. The length dimension of any or all of the face surface, the flanged tissue engagement surface and the head may be larger than a first exterior width of the conduit defined by an extent of the protrusion areas transverse to the length of the implant device, when the implant device includes an anchoring surface feature such as summarized above. The length dimension of any or all of the face surface, the tissue engagement surface and the head may be in a range having a lower limit of 1 millimeter, 2 millimeters, 2.5 millimeters, 2.75 millimeters, 3 millimeters, 3.5 millimeters, 4 millimeters or 5 millimeters and an upper limit of 10 millimeters, 8 millimeters or 7 millimeters. The width dimension of any or all of the face surface, tissue engagement surface and the head may be in a range having a lower limit of 0.5 millimeter, 1 millimeter, 1.5 millimeters, 1.75 millimeters or 2 millimeters and an upper limit of 5 millimeters, 4 millimeters, 3 millimeters, 2.5 millimeters or 2 millimeters, provided as always that the upper limit is larger than the lower limit. The length dimension of any or all of the face surface, the flanged tissue engagement surface and the head may be at least 0.5 millimeters, at least 0.75 millimeter, at least 1 millimeter, at least 2 millimeters, at least 3 millimeters or at least 4 millimeters larger than such first exterior width of the conduit defined by an extent of the protrusion areas, when the implant device includes an anchoring surface feature such as summarized above. A ratio of the length of any of or all the face surface, the flanged tissue engagement surface and the head to such a first exterior width of the conduit may be at least 1.5 or at least 2. Such a ratio may be smaller than 4, smaller than 3 or smaller than 2.5. The width of any or all of the face surface, the flanged tissue engagement surface and the head may be not larger than, or may be smaller than (e.g., by at least 0.1 mm or by at least 0.2 mm), such a first exterior width of the conduit defined by an extent of the protrusion areas, when the implant device includes an anchoring surface feature such as summarized above. A ratio of the length dimension to the width dimension for any or all of the face surface, the flanged tissue engagement surface and the head may be in a range having a lower limit of 1, 1.25, 1.5, 2 or 2.5 and an upper limit of 5, 4, 3 or 2.5, provided of course that the upper limit must be larger than the lower limit. Having a larger length dimension to width dimension on the head is preferred in some implementations when the head will be located in the orbit between an eyeball and a medial corner of the palpebral fissure (e.g., between the lacrimal caruncle and the plica semilunaris or through the lacrimal apparatus), because the length dimension may advantageously align in a vertical direction next to the eyeball and will help provide sufficient flanged surface area to effectively anchor the implant device on the proximal end and impede conjunctival tissue from covering the opening into the internal passage of the implant device, compensating for the narrower width. This is particularly advantageous when using polymeric materials of construction as described above.

The lacrimal apparatus and a paranasal sinus may be in fluid communication through the internal passage of the implant device when the implant device is implanted. The conduit may extend from adjacent the proximal end of the implant device. The conduit may extend to adjacent the distal end of the implant device. The internal passage may have a first end open at the proximal end and a second end open at the distal end, and when the implant device is implanted the first end of the internal passage may open in the lacrimal apparatus and the second end of the internal passage may open in the paranasal sinus.

The implant device may be configured for implantation with the conduit passing through a fistula between a location in a lacrimal apparatus within the orbit and a paranasal sinus selected from the group consisting of a frontal sinus, an ethmoid sinus, a maxillary sinus and a sphenoid sinus, with a frontal sinus, a maxillary sinus or an ethmoid sinus being preferred, with an ethmoid sinus or a maxillary sinus being more preferred, and with an ethmoid sinus being particularly preferred. The implant device may be configured for the conduit to pass through a first paranasal sinus and into a second paranasal sinus in which the distal end of the implant device is disposed when implanted. The implant device may provide fluid communication between the lacrimal apparatus and the second paranasal sinus and may or may not also provide fluid communication between the lacrimal apparatus and the second paranasal sinus. For example, the portion of the conduit passing through the first paranasal sinus may have an impermeable wall to prevent fluid communication with the first paranasal sinus or may have one or more openings through the wall that provide permeability through the wall and a fluid path for fluid communication to the first paranasal sinus, in which case fluids (e.g., treatment formulations) may be introduced into either one or both of the first and second paranasal sinuses. As one example, an implant device may be configured to be implanted with a proximal end in the lacrimal apparatus in the orbit and a distal end in an ethmoid sinus with the conduit passing through a frontal sinus or a maxillary sinus.

Various feature refinements and additional features are applicable to other methods.

A method may be or involve administering a treatment composition to a paranasal sinus through an implant device of the first aspect that fluidly connects the lacrimal apparatus with the paranasal sinus.

Various feature refinements and additional features are applicable to the packaged product aspect and the method aspect involving implanting an implant device of such a packaged product.

The implant device of the product may include any feature or combination of features disclosed in relation to the first aspect or any other aspect. The implant device may be according to the first aspect, or may not be according to the first aspect. The implant device may be according to the first aspect including feature (xvi).

The storage liquid may provide for enhanced wettability of some or all exterior surfaces of the implant device (e.g., surfaces other than the surfaces of the walls of an internal passage within the implant device). In some implementations, all exterior surfaces of the implant device may be in contact with the storage liquid. Internal passage surfaces may also be in contact with the storage liquid. Substantially all surfaces of the implant device may be in contact with the storage liquid. The implant device may be immersed in a reservoir of the storage liquid.

The storage liquid may comprise an aqueous liquid, which may be a buffer solution. The storage liquid may include a wetting agent, which may improve hydration of at least a portion of exposed surfaces of the implant device following implantation. In some implementations, the implant device includes a head disposed in the lacrimal apparatus when the implant device is implanted, and the wetting agent may improve hydration of exterior surfaces of the head within the lacrimal apparatus. In some implementations, the implant device may include a distal portion disposed in the paranasal sinus when the implant device is implanted, and the wetting agent may improve hydration of exterior surface of such distal portion following implantation. A wetting agent may include any material that provides enhanced wettability of any such exterior surfaces (e.g., of a head or a distal portion of the implant device) to wetting by biological fluid, for example wetting of head surfaces by lacrimal fluid. The wetting agent may be any wetting agent, for example as discussed above. On preferred wetting agent is hyaluronic acid.

The sterile packaging may include any sterile barrier enclosure, for example in the form of a sealed bag, pouch or tray. A packaged product may include a plurality of such implant devices, with the sterile packaging including a plurality of sealed compartments each having disposed therein an implant device in contact with storage liquid. The sterile packaging may include a multi-compartment tray comprising such an implant device sealed within each of a plurality of such compartments of the tray. Each such compartment may be sealed so that each such sealed compartment is individually unsealable to remove an implant device from a compartment without unsealing other compartments. In some implementations, the sterile packaging may include a multi-compartment tray (e.g., of a plastic material) with compartments sealed with a foil lid.

During a method for implanting an implant device from a packaged product, the implant device may have at least a residual portion of the storage liquid, which may include at least a portion of a wetting agent, covering at least a portion of the implant device.

Other feature refinements and additional features are presented in the example implementation embodiments and the claims presented below.

These and other aspects of the disclosure, and possible feature refinements and additional features therefore, will be further understood with reference to the drawings, to the description provided below and to the claims.

### BRIEF DESCRIPTION OF THE DRAWINGS

The drawings are included to aid in the understanding of various aspects of the disclosure and possible feature refinements and additional features applicable thereto. Features shown in the drawings are presented for purposes of illustration only, and are not necessarily to scale and are not necessarily detailed in every respect.

FIG. 1 is an illustration showing some example routes for an implant to provide fluid access from the lacrimal apparatus to a paranasal sinus.

FIG. 2 is a side view of an embodiment of a paranasal sinus access implant device.

FIG. 3 is a perspective view of the embodiment of a paranasal sinus access implant device of FIG. 2.

FIGS. 4 and 5 are sectional views of a proximal end portion of the embodiment of a paranasal sinus access implant device of FIGS. 2 and 3.

FIGS. 6 and 7 are sectional views of a distal end portion of the embodiment of a paranasal sinus access implant device of FIGS. 2 and 3.

FIG. 8 shows perspective, top, side and end views of an embodiment of an implant device.

FIG. 9 is a side view of an embodiment of an implant device.

FIG. 10 is a side view of an embodiment of an implant device.

FIG. 11 is a side view of an embodiment of a preliminary implant device form.

FIG. 12 is a side view of an embodiment of a modified implant device form prepared by modification of the preliminary implant device form of FIG. 11.

FIG. 13 is a side view of a further modified implant device form prepared by modification of the modified implant device form of FIG. 12.

FIG. 14 is a sectional view of a portion of the head of the modified implant device form of FIG. 13.

FIG. 15 is a side view of a further modified implant device form prepared by modification of the modified implant device form of FIG. 13.

FIG. 16 is a sectional view through a portion of the conduit of the modified implant device form of FIG. 15.

FIG. 17 is a further modified implant device form prepared by modification of the modified implant device form of FIG. 15.

FIG. 18 is sectional view of an embodiment of a preliminary implant device form.

FIG. 19 is a sectional view of an embodiment of a modified implant device form prepared by modification of the preliminary implant device form of FIG. 18.

FIG. 20 is a sectional view of an embodiment of an implant device.

FIGS. 21 and 22 are side views of an embodiment of an implant device including a conduit extension portion in a fully contracted and fully extended configuration, respectively.

FIGS. 23-28 illustrate another embodiment of an implant device.

FIG. 29 is an illustration showing an embodiment for implant placement of an implant device with a head of the implant device located over the lacrimal caruncle in the lacrimal apparatus in the orbit.

FIG. 30 is an illustration showing use of a surgical tool, in the form of a carrier tool, for implantation of an implant device during a surgical procedure.

FIG. 31 is an illustration showing placement of an implant device following implantation during a surgical procedure.

FIG. 32 is a generalized process block diagram of an example generalized processing to make an example embodiment of a paranasal sinus access implant device.

FIGS. 33-36 are illustrations of example processing operations for some of the generalized processing steps of FIG. 32.

### DETAILED DESCRIPTION

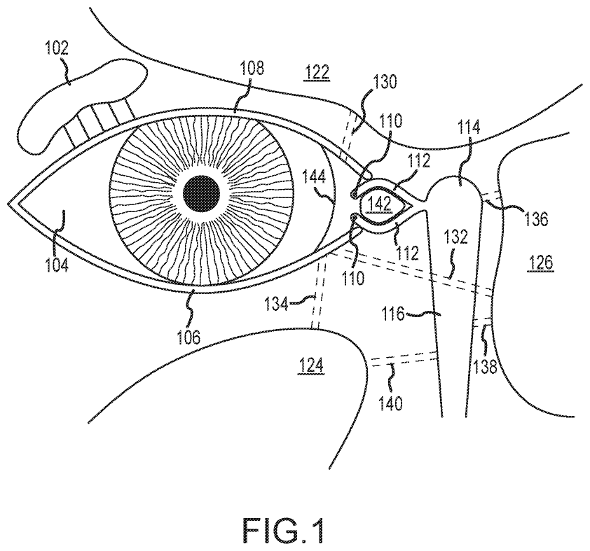

The terms “lacrimal apparatus” and “lacrimal system” are used interchangeably herein to refer to the collection of physiological components that accomplish the production and secretion of lacrimal fluid to lubricate the eyeball, containment of lacrimal fluid in a reservoir of lacrimal fluid in the orbit and drainage of lacrimal fluid from the orbit to the nasal cavity. The lacrimal apparatus includes the lacrimal glands, the tear drainage system and the reservoir of lacrimal fluid located between the lacrimal glands and the tear drainage system. The reservoir of lacrimal fluid includes the eyelid margins and the conjunctival sac (and including the pool of tears in the lower conjunctival cul-de-sac that is sometimes referred to as the lacrimal lake). The tear drainage system includes the puncta, canaliculi and nasolacrimal duct (including the so-called lacrimal sac located at the top of the nasolacrimal duct) through which excess tears drain to Hasner's valve and into the nasal cavity. FIG. 1 shows generally the lacrimal apparatus. Lacrimal fluid is produced and secreted from lacrimal glands 102 to lubricate the surface of the eyeball 104 disposed within the orbit. Lacrimal fluid forms a coating over the eyeball 104 and is generally contained within the conjunctival sac (the space between the lower eyelid 106, upper eyelid 108 and eyeball 104 that is lined by the conjunctiva). Excess lacrimal fluid is conducted to the vicinity of the medial canthus (medial corner of the eye) and drains through the lacrimal puncta 110 into the lacrimal canaliculi 112 and into the lacrimal sac 114 of the nasolacrimal duct 116. The lacrimal fluid then drains from the nasolacrimal duct 116 through Hasner's valve and into the nasal cavity.

As used herein, a fistula between the lacrimal apparatus and a paranasal sinus refers to an artificially-created passage that fluidly connects the lacrimal apparatus with a paranasal sinus. Such a fistula may be surgically created. The paranasal sinuses include the frontal sinuses, maxillary sinuses, ethmoid sinuses and sphenoid sinuses, which are cavities contained within frontal, maxilla, ethmoid and sphenoid bones, respectively. The paranasal sinuses drain into the nasal cavity. FIG. 1 also shows the general proximity of the frontal sinus 122, maxillary sinus 124 and ethmoid sinus 126 relative to features of the lacrimal apparatus and some example fistula routes shown by dashed lines. A first example fistula route 130 is from the orbit to the frontal sinus. A second example fistula route 132 is from the orbit to the ethmoid sinus 126. A third example fistula route 134 is from the orbit to the maxillary sinus 124. A fourth example fistula route 136 is from the lacrimal sac 114 at the top of the nasolacrimal duct 116 to the ethmoid sinus 126. A fifth example fistula route 138 is from the nasolacrimal duct 116 at a location below the lacrimal sac 114 to the ethmoid sinus 126. A sixth example fistula route 140 is from the nasolacrimal duct 116 at a location below the lacrimal sac 114 to the maxillary sinus 124. The example fistula routes shown in FIG. 1 are for purposes of general illustration only and not to show precise locations where a fistula might be formed to connect a part of the lacrimal apparatus with the corresponding paranasal sinus. Although not shown in FIG. 1, example fistula routes to the sphenoid sinus include from the orbit to the sphenoid sinus and from the nasolacrimal duct 116 to the sphenoid sinus.

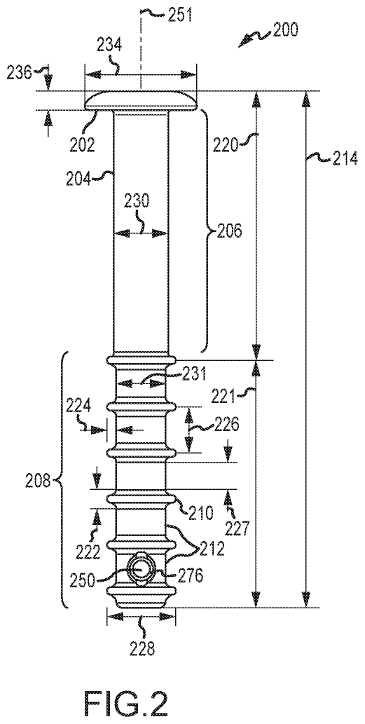

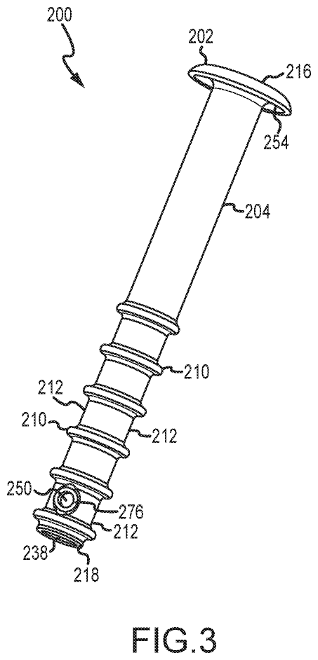

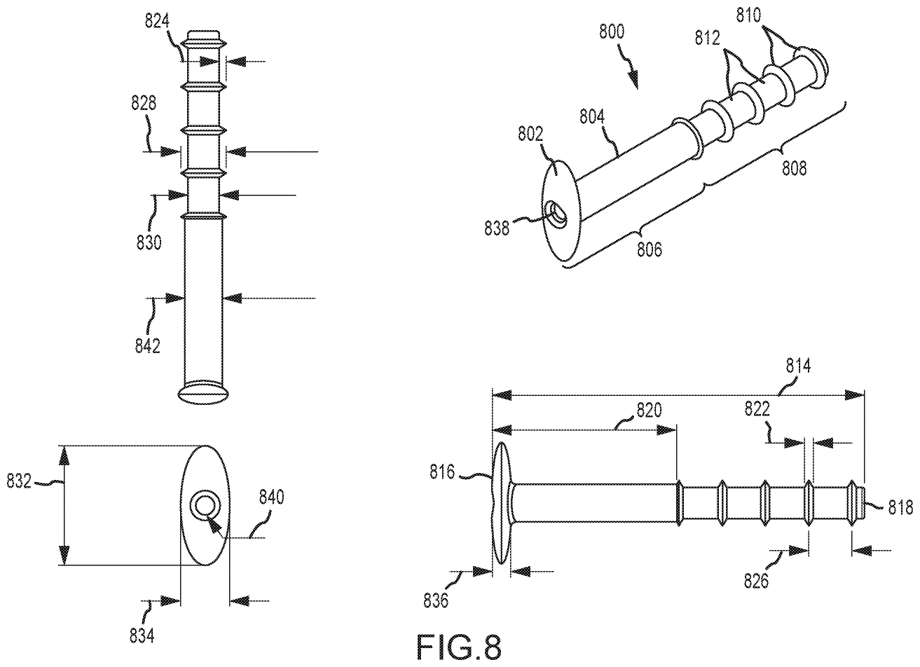

FIGS. 2 and 3 show an implant device 200 with a head 202 and a conduit 204. The conduit 204 includes a first longitudinal portion 206 and a second longitudinal portion 208 disposed distal of the first longitudinal portion 206. The first longitudinal portion 206 includes a smooth exterior surface and second longitudinal portion 208 includes an anchoring surface feature including anchoring protrusions in the form of spaced circumferential ridges 210 and recess areas 212 between the ridges 210. The length of the first longitudinal portion 206 of the conduit 204, which includes the portion of the conduit 204 located proximal to the first ridge 210, may advantageously be disposed through conjunctival tissue adjacent the head 202 when implanted to “float” for patient comfort and to facilitate performance of post-implantation medical procedures without disruption of implant anchor stability. The anchoring features of the second longitudinal portion 208 may advantageously be located a distance from the head 202 so that one or more of the ridges 210 are located in the vicinity of the paranasal sinus bone wall that is penetrated by the implant device 200 when implanted, preferably with one or more of the anchor protrusions disposed on each side of the bone. Preferably, the wall of the sinus cavity bone penetrated by the implant device 200 is disposed between two adjacent ones of the ridges 210. The implant device 200 shown in FIGS. 2 and 3 has a different exterior width of the conduit 204 in the first longitudinal portion 206 than in the recess areas 212 of the second longitudinal portion 208. The implant device 200 includes an internal passage 238 extending between the proximal end 216 and the distal end 218, passing through the head 202 and the full length of the conduit 204. Similar to the implant device 800 shown in FIG. 8 discussed below, the implant device 200 of FIGS. 2 and 3 includes a thicker wall of the conduit adjacent the internal passage 238 in the first longitudinal portion 206 of the conduit than in the recess areas 212 of the second longitudinal portion 208. The internal passage 238 is open at the proximal end 216 for fluid communication with the lacrimal apparatus when implanted and is open at the distal end 218 for fluid communication with a paranasal sinus when implanted, whereby the implant device 200 when implanted provides a fluid communication path between the lacrimal apparatus and the paranasal sinus. Disposed between the most distal pair of adjacent ridges 210, the implant device 200 includes two side ports 250 disposed on opposite sides of a longitudinal axis 251 of the implant device 200.

Various dimensions are shown in FIG. 2 for the implant device 200, in addition to the length 214. The circumferential ridges 210 have a width 222 at the base of the ridges 210 and a height 224 above adjacent recess areas 212. The ridges 210 are spaced on a center-to-center spacing 226, with inter-ridge spacing 227 between adjacent bases of adjacent ridges 210. The conduit 204 has a maximum exterior width 228 corresponding with the tops of the ridges 210, equal to the diameter of the circle of the cross-section through the conduit 204 at the top of the ridges 210. The conduit 204 has a minimum exterior width 231 at locations corresponding with the recess areas 212 on the second longitudinal portion 208 of the conduit 204. The head 202 has a circular perimeter having a diameter 234 and a depth 236. The beginning, or proximal end, of the second longitudinal portion 208 is located at a distance 220 from the proximal end 216, at the base of the ridge 210 nearest to the proximal end 216 and the second longitudinal portion 208 has a length 221. The internal passage 238 has a circular cross-section along the length of the implant device 200, which is of constant diameter except that the diameter of the internal passage flares to a larger diameter in transition portions adjacent the proximal end 216 and the distal end 218, which are further described below.

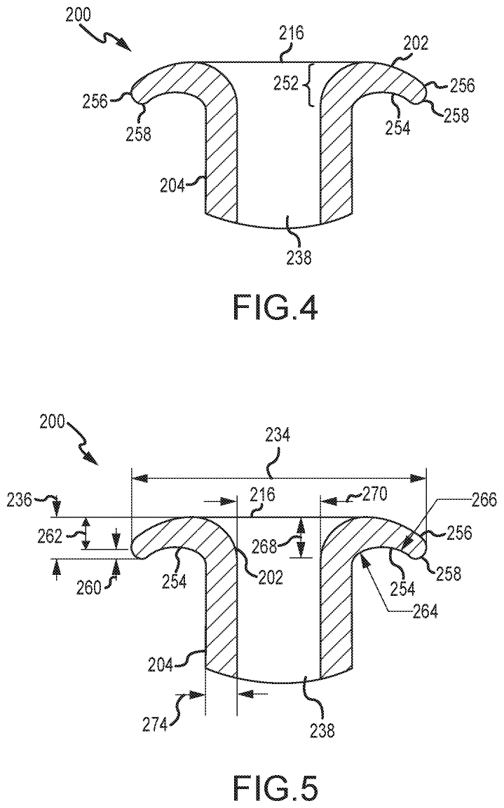

Reference is now made to FIG. 4 in combination with FIGS. 2 and 3. FIG. 4 shows a proximal portion of the implant device 200 in cross-section, including the head 202 adjacent the proximal end 216 and the conduit 204 extending from the head 202 in the distal direction. As shown in FIG. 4, the internal passage 238 includes a proximal port transition portion 252 adjacent the proximal end 216 in which the diameter of the internal passage 238 flares towards the proximal end 216. In the proximal port transition portion 252, the diameter of the internal passage 238 flares, or becomes larger, in a direction from the distal end 218 toward the proximal end 216, or conversely has a tapering cross-section that tapers, or becomes smaller, in a direction from the proximal end 216 toward the distal end 218 of the implant device 200.

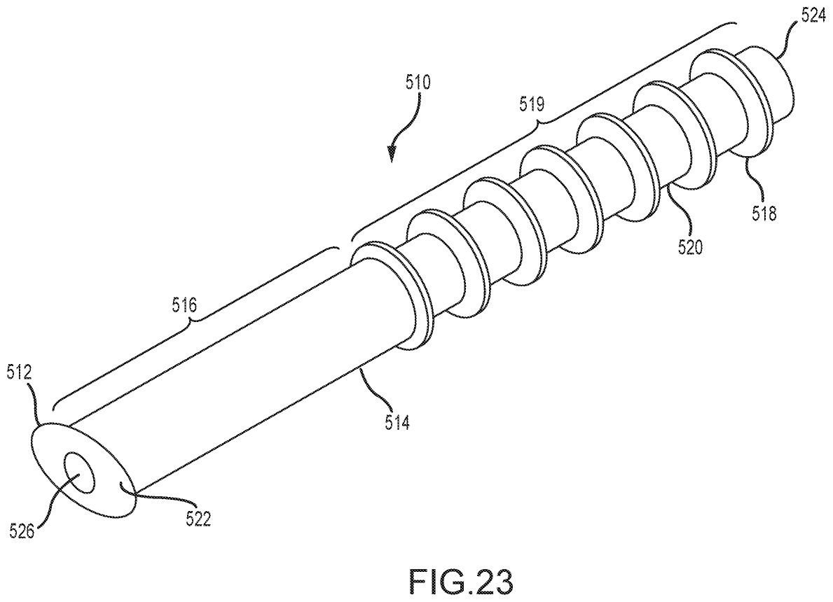

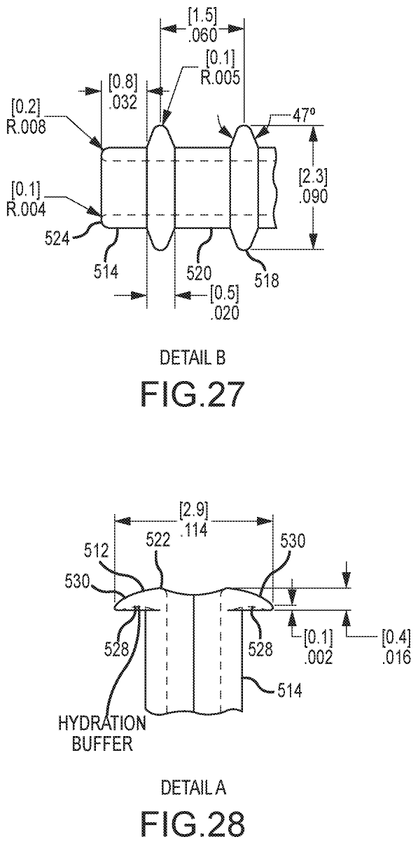

Another important feature shown in FIG. 4 is the presence of a concave undercut portion 254 on a distal side of a flanged portion 256 of the head 202. By a flanged portion, it is meant a portion of the head 202 that extends radially outward relative to the outside diameter of the conduit 204 adjacent to the head 202. By a distal side of the flanged portion 256, it is meant the side of the flanged portion 256 that is disposed toward the distal end 218 of the implant device 200 and by a proximal side of the flanged portion 256 it is meant the side of the flanged portion 256 opposite the distal side, and which is disposed toward the proximal end 216 of the implant device 200. Generally, the distal side of flanged portion 256 will be disposed toward and engage tissue in the lacrimal apparatus adjacent to the fistula through which the conduit 204 extends when the implant device 200 is implanted and the proximal side of the flanged portion 256 generally faces away from such tissue. The concave undercut portion 254 is a hollowed-out region that recesses into the flanged portion 256 relative to a distal lip 258 projecting from the distal side of the flanged portion 256. The concave undercut portion 254 provides several advantages. The recess area provides a location for accumulation and retention of moisture to maintain good hydration of tissue adjacent the distal side of the flanged portion 256. The concave undercut portion 254 also has a curved geometry configured to better conform with the natural surface geometry of tissue against which the distal side of the flange portion 256 will be disposed when the implant device 200 is implanted with the head 202 disposed in the lacrimal apparatus in the orbit. More particularly, the geometry of the concave undercut portion 254 may generally correspond with the curved geometry of the lacrimal caruncle. It has been found to be convenient to implant paranasal sinus access implant devices from the lacrimal apparatus in the orbit by forming a fistula through the lacrimal caruncle and implanting the implant device so that flanged portions of the head of the implant device are in contact with the portions of the lacrimal caruncle surrounding the fistula. Similar advantages are provided by the corresponding concave undercut portion in the implant device 510 shown in FIGS. 23-28, discussed below. In the embodiment of the implant device 510 shown in FIGS. 23-28, separate concave undercut portions are provided on each of the opposing flanged portions of the head 512 by the concave surfaces 528. In general, having a single concave undercut that extends 360° around the longitudinal axis within the parameter of the head, as illustrated in FIGS. 2-4, is a preferred feature for enhanced conformance with the geometry of the lacrimal caruncle.

Reference is now made to FIG. 5 in combination with FIGS. 2-4. FIG. 5 shows the same sectional view of a proximal portion of the implant device 200 as shown in FIG. 4. FIG. 5 shows various dimensions for a contemplated implementation example of the proximal portion of the implant device 200. FIG. 5 shows the diameter 234 of the circular perimeter of the head 202 and the depth 236 of the head 202, shown also in FIG. 4. As best as seen in FIG. 5, the depth 236 of the head 202 is the longitudinal distance between the point on the head 202 that projects farthest in the proximal direction, which for the embodiment shown in FIGS. 4 and 5 corresponds with the proximal end 216, and the most distal projection of the head, which in the embodiment shown in FIGS. 4 and 5 corresponds with the distal lip 258. Also shown in FIG. 5 is the depth 260 of the concave undercut 254, which represents the maximum recess projection of the concave undercut portion 254 relative to the most distal point on the flanged portion 256, which corresponds with the distal lip 258 on the flanged portion 256. Also shown is the flange thickness 262 of the flanged portion 256 corresponding with thickness of the flanged portion 256 at the location of the maximum recess projection within the concave undercut portion 254. The particular geometry of the concave undercut portion 254 as shown in FIGS. 4 and 5 includes a first curved geometry with a first radius of curvature 264 and a second curved geometry with a second, larger radius of curvature 266. Also shown is the longitudinal length 268 of the proximal port transition portion 252, which is the longitudinal distance into the internal passage 238 to which the proximal port transition portion 252 extends, and the tapered diameter 270 of the internal passage 238 at the distal end of the proximal port transition portion 252. In the embodiment shown in FIGS. 2-5, the diameter of the circular cross-section of the internal passage 238 remains constant at the tapered diameter 270 down the conduit 204 in the distal direction along the implant device 200 until a corresponding distal port transition portion adjacent to the distal end 218 of the implant device 200, discussed below. Also shown in FIG. 5 is the wall thickness 274 of the conduit 204 in the first longitudinal portion 206 of the conduit 204 located distal of the proximal port transition portion 252.

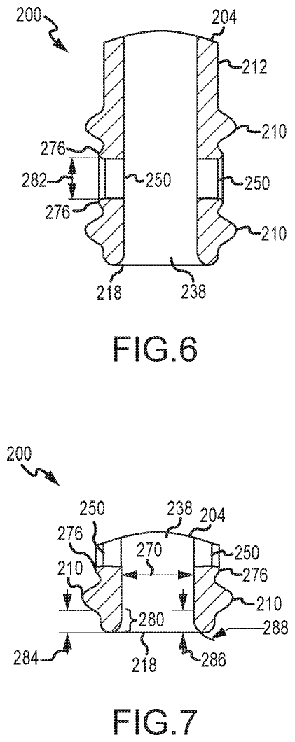

Reference is now made to FIGS. 6 and 7 in combination with FIGS. 2-5. Each of FIGS. 6 and 7 shows a sectional view of a distal portion of the implant device 200 of FIGS. 2-5, with part of the sectional view of FIG. 6 being also shown in FIG. 7 to facilitate description of certain features of the portion of the implant device 200 adjacent the distal end 218. FIGS. 6 and 7 show the side ports 250 located between the most distal pair of adjacent circumferential ridges 210. For added structural rigidity in the vicinity of the side ports 250 and to facilitate convenient manufacture, the side ports 250 are encircled by a circular ridge 276 (also shown in FIGS. 2 and 3). The circular ridge 276 protrudes slightly radially outward relative to the recess area 212 between the distal pair of adjacent ridges 210. Although such side ports 250 may be formed during a molding operation to manufacture the implant device 200, molding may be simplified if the side ports 250 are punched through the wall of the conduit 204 following molding. The raised feature of the circular ridge 276 provides a convenient feature for properly orienting the implant device 200 with punching equipment for proper punching of the side ports 250.

FIGS. 6 and 7 also show a distal port transition portion 280 of the interior passage 238. In the distal port transition portion 280, the diameter of the internal passage 238 flares out relative to the tapered diameter 270 of the internal passage 238. In that regard, the cross-section of the internal passage 238 in the distal port transition portion 280 tapers in a direction from the distal end 218 toward the proximal end 216 of the implant device 200. The flaring nature of the internal passage in the proximal port transition portion 252 and the distal port transition portion 280 toward the proximal end 216 and distal end 218, respectively, help to prevent buildup of debris at the fluid communication ends of the internal passage 238. Some example dimensions are also shown in FIGS. 6 and 7, including the tapered diameter 270 of the internal passage 238 and the diameter 282 of the side ports 250. Also shown is the setback 284 of the last ridge 210 from the distal end 218 of the implant device 200 and the length 286 of the distal port transition portion along the length of the implant device 200. Also shown is the radius of curvature 288 of the curved geometry of the flaring wall of the conduit 204 in the internal passage 238 in the distal port transition portion 280.

Some example values for a number of the dimensions shown in FIGS. 2-7 for one specific example implementation contemplated for an example implementation of the implant device 200 are summarized in Table 1.

TABLE 1

| Dimension | |
|---|---|
| of Implant Device | Specific Example |
| 214 | 17.8 mm |
| 220 | 8 mm |
| 221 | 8.9 mm |
| 222 | 0.44 mm |
| 224 | 0.3 mm |
| 226 | 1.65 mm |
| 227 | 1.21 mm |
| 228 | 2.41 mm |
| 230 | 2 mm |
| 231 | 1.8 mm |
| 234 | 4 mm |
| 236 | 0.9 mm |
| 260 | 0.18 mm |
| 262 | 0.71 mm |
| 264 | 0.25 mm |
| 266 | 0.58 mm |
| 268 | 0.9 mm |
| 270 | 1.14 mm |
| 274 | 0.43 mm |
| 282 | 0.64 mm |
| 284 | 0.32 mm |
| 286 | 0.32 mm |
| 288 | 1.27 mm |

FIG. 8 shows an implant device 800 that is similar in many respects to the implant device 200 shown in FIGS. 2-7, including a first longitudinal portion of a conduit having a thicker wall than recess areas of the anchoring surface feature of a second longitudinal portion of the conduit. As with the implant device 200 of FIGS. 2-7, the thicker wall in the first longitudinal portion of the conduit provides added rigidity to that portion of the conduit to facilitate pushing the implant device 800 into place during an implantation procedure, while the thinner wall in the recess areas of the second longitudinal portion of the conduit permit that portion to more easily deform and fit through a fistula during implantation and then to expand to engage tissue and anchor the implant device 800. More specifically as shown in FIG. 8, the implant device 800 includes a head 802 and a conduit 804. The conduit 804 has a first longitudinal portion 806 and a second longitudinal portion 808 located distal of the first longitudinal portion 806. The first longitudinal portion 806 includes a substantially smooth exterior surface with a substantially constant exterior width, which is the diameter of the circular cross-section of the conduit 804 along the first longitudinal portion 806. The second longitudinal portion 808 includes an anchoring surface feature including anchor protrusions 810, in the form of circumferential ridges, and recess areas 812 in the spaces between the anchor protrusions 810. Various dimensions of the implant device 802 are illustrated in FIG. 8, similar to the illustration provided for the implant device 200 in FIGS. 2-7. The implant device 800 has a length 814 from a proximal end 816 to a distal end 818 of the implant device 800. The beginning of the second longitudinal portion 808 is located a distance 820 distal of the proximal end 816. The anchor protrusions have a width 822 at the base and a height 824 above the adjacent recess areas 812. The anchor protrusions are spaced on a center-to-center spacing 826. The conduit 804, and also the second longitudinal portion 808, has a maximum exterior width 828 occurring at the tops of the anchor protrusions 810, and equal to the diameter of the circular cross-section of the conduit 804 through the tops of the anchor protrusions 810. The conduit 804, and the second longitudinal portion 808, of the conduit 804 have a minimum exterior width 830 located at the recess areas 812. The head 802 has a length dimension 832, a width dimension 834 and a depth dimension 836. The head 802 has an elongated shape with a longer length dimension 832 than the width dimension 834. The implant device 800 has an internal passage 838 extending between the proximal end 816 and the distal end 818 and through the length of the conduit 804. The internal passage 838 has a width 840, which in the embodiment shown in FIG. 8 is equal to a diameter of the circular cross-section of the internal passage 838.

With continued reference to FIG. 8, similar to the implant device 200 of FIGS. 2-7, in the implant device 800 the wall thickness of the conduit 804, (thickness of the wall between the internal passage 838 and the exterior surface of the conduit 804) is greater along the first longitudinal portion 806 than in the recess areas 812 of the second longitudinal portion 808. The internal passage 838 has a constant width along the length of the conduit 804, such that the greater wall thickness of the conduit 804 along the first longitudinal portion 806 results in an exterior width 842 that is larger than the minimum exterior width 830 in the recess areas 812. The maximum exterior width 828 at the anchor protrusions 810 is larger than the exterior width 842 along the first longitudinal portion 806.

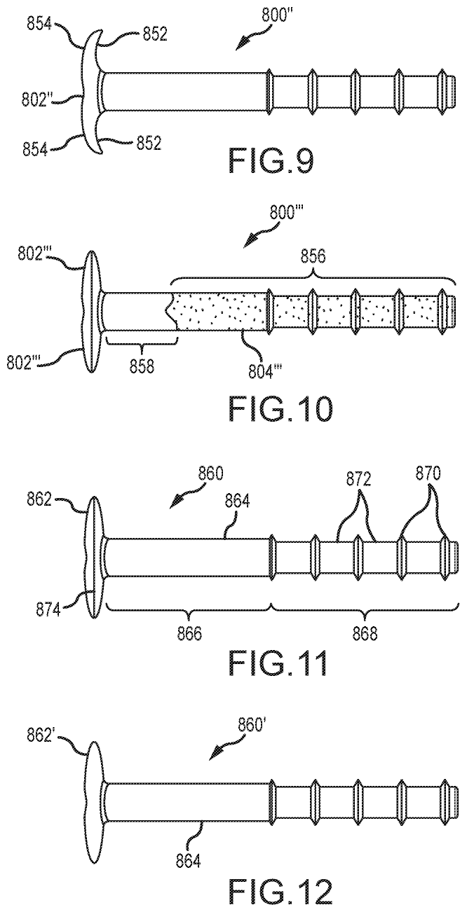

FIG. 9 shows an example variation on the implant device 800 of FIG. 8. The implant device 800″ variation shown in FIG. 9 has the same general features as the implant device 800 of FIG. 8, but with a different head design. The head 802″ shown in FIG. 9 has flanged portions with a distal side having concave surfaces 852 disposed towards the distal end of the implant device 800″. The flanged portions of the head 802″ have a proximal side with convex surfaces 854 disposed away from the distal end of the implant device 800″. The concave surfaces 852 may provide an advantage of better conforming with soft tissue within the orbit engaged by the distal side of the flanged head portions when the implant device 800″ is implanted to fluidly connect the lacrimal apparatus in the orbit with a paranasal sinus. The convex surfaces 854 also may better conform with the surface of such soft tissue when the implant device 800″ is implanted. The head 802″ may thus help reduce possibility for a foreign body sensation when the implant device 800″ is implanted.

FIG. 10 shows another example variation on the implant device 800 of FIG. 8. The implant device 800″ variation shown in FIG. 10 has the same general features as the implant device 800 of FIG. 8, but includes a conduit 804′″ including a radiopaque portion 856 extending over a significant length of the conduit 804″. The conduit 804′″ also includes a radiotransparent portion 858 located adjacent the proximal end of the conduit 804′″. The head 802′″ is also radiotransparent. The head 802′″ in particular is preferably clear and substantially transparent or translucent to visible light so that when the implant device 800″ is implanted with the head 802′″ disposed in the orbit, the head 802′″ is not highly visible. In contrast, the radiopaque portion 856 of the conduit 804′″ may be easily imaged by radiation, for example by x-ray imaging, to verify the positioning of the conduit 804′″ in a patient's body when the implant device 800′″ is implanted or at any time post implantation, for example to verify proper implant placement. As an alternative to the variation shown in FIG. 10, such an implant device could be made to be entirely radiopaque, however such a variation may be not preferred for some applications due to enhanced visibility of the head 802″, which may be generally less aesthetically pleasing to patients in which the implant device is implanted. The implant device 800′″ may be made, for example, by introducing a radiotransparent resin composition into a proximal end of a mold and a radiopaque resin composition into a distal end of the mold, with an interface between the different resin compositions forming at an intermediate location in the mold based on the relative proportions of the different resin compositions introduced into the mold. For example, such a radiopaque portion may be a resin composition including a radiopaque agent (e.g., particulate radiopaque filler), while the radiotransparent composition may include the same or a different resin (e.g., silicone or polyurethane resin).

Reference is now made to FIGS. 11-17 in relation to various examples of methods of making a paranasal sinus implant device in which a preliminary form is modified to make a modified implant device form, which modified implant device form may be a final implant device product or may be a new preliminary form to be further processed in the manufacture of a further intermediate form or a final product. FIG. 11 shows a preliminary form 860 including a preliminary head structure 862 and a preliminary conduit structure 864. The preliminary conduit structure 864 includes a first longitudinal portion 866 and a second longitudinal portion 868. First longitudinal portion 866 has a substantially smooth exterior surface with a substantially constant exterior width of circular cross-section. The second longitudinal portion 868 includes an anchoring surface feature including anchor protrusions 870, in the form of circumferential ridges, and recess areas 872 in the spaces between the anchor protrusions 870. The first longitudinal portion 866 of the preliminary conduit 864 may have a larger minimum wall thickness than the second longitudinal portion 808. The preliminary conduit 864 is illustrated with features substantially as described with respect to the conduit of the implant device shown in FIG. 8, but could be of a different design, for example with one or more features shown in any of FIG. 2-7, 9, 10, or 18-28. The preliminary head structure 862 is also illustrated with features substantially as shown in FIG. 8, but could be of a different design, for example with one or more features shown in any of FIG. 2-7, 9, 10, or 18-28. As shown in FIG. 11, the preliminary head structure 862 includes flash 874 around the perimeter of the preliminary head 862. For example, the preliminary implant device form 860 may be an initial mold product (e.g., from injection molding, compression molding or transfer molding) of one or more resin compositions (e.g., silicone or polyurethane resin), and the flash 874 may be formed at a joint between parts of the mold. Presence of such flash 874 may contribute to a patient experiencing a foreign body sensation when implanted. Such flash 874 may be removed to smooth the surface of the head and reduce potential for such a foreign body sensation and may also help to reduce potential for biofilm formation on the smoother surface. Such removal of the flash 874 may be effected by any removed technique, for example by trimming, cryogenic tumbling or laser ablation.

FIG. 12 shows a modified implant device form 860′ with a modified head structure 862′ after removal of the flash 874 from the preliminary implant device form 860 of FIG. 11. The preliminary conduit 864 is unaltered in FIG. 12. The modified implant device 860′ may be used as a final implant device product, or may be used as a further preliminary form that is further modified. For example, following removal of the flash 874, the modified head structure 862′ may be further modified by forming a thin layer of softer material over the modified head structure 862′, for example a layer of a silicone material that is softer than the initial structural material of the preliminary head structure 862. Such a layer of softer material may be formed, for example, by dip molding or spray molding a softer layer of material on top of the modified head structure 862′.

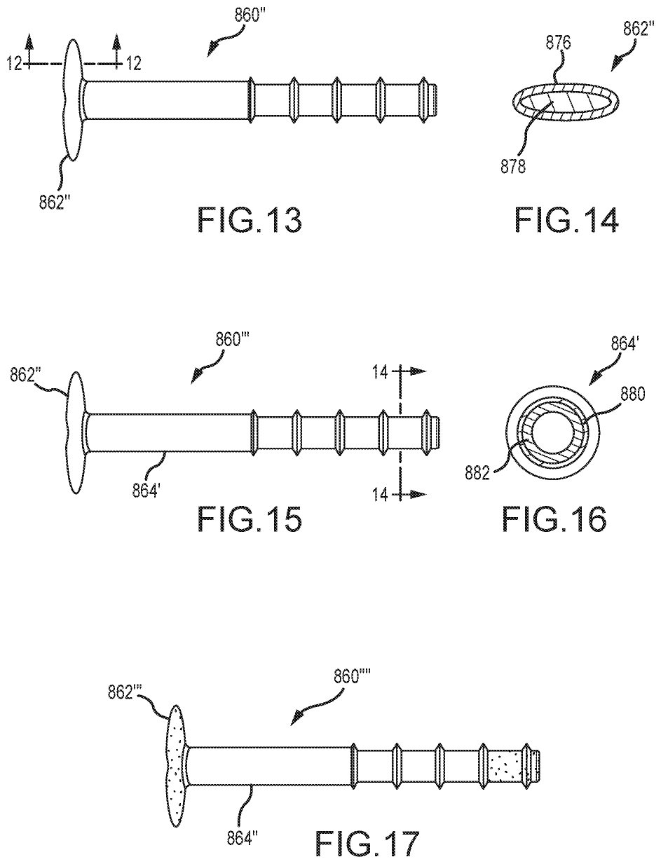

FIG. 13 shows an example following formation of such a softer material layer, with the implant device form now designated as a further modified form 860″. The modified implant device form 860″ includes a modified head structure 862″ that is slightly enlarged by addition of such a thin layer of softer material. As shown in the sectional view of FIG. 14, the modified head structure 862″ includes a layer of softer material 876 formed over original structural material 878 of the original head form 862 of FIG. 11. The presence of the softer material 876 may further enhance compatibility with soft tissue within the orbit when implanted, which may further help to avoid a foreign body sensation following implantation. Moreover, such a carefully applied layer of softer material 876, such as may be formed by dip coating, may have a significantly smoother exterior surface than the exterior surface of the original preliminary head structure 862 of FIG. 11 or the modified head structure 862′ of FIG. 12. Such smoother exterior surface may further assist in reducing potential for a foreign body sensation following implantation and may also reduce potential for biofilm formation on exposed surfaces of the modified head structure 862″. The modified implant device form 860″ may be used as a final implant device product, or may be used as a further preliminary form that is further modified.

In a similar manner to the formation of the layer of softer material 876 shown in FIGS. 13 and 14, the conduit may be similarly modified by forming a thin layer of softer material over a portion of the preliminary conduit structure 864. FIG. 15 shows a further modified implant device form 860′″ including a modified conduit structure 864′ including such a layer of softer material formed over a distal portion of the initial preliminary conduit structure 864 shown in FIG. 11 As shown in the sectional view of FIG. 16, the modified distal portion of the modified conduit structure 864′ includes a layer of softer material 880 over the original structural material 882 of the initial preliminary conduit structure 864. Such a layer 880 could, for example, be of a silicone material that is softer than the initial structural material of the preliminary conduit structure 864. Such a layer of softer material 880 may advantageously be provided only on a distal portion of the conduit that extends into the paranasal sinus when the implant device is implanted, although such a softer layer could be placed over other portions of the conduit as well if desired. In some preferred implementations, the harder structural material 882 would remain exposed along the portion of the conduit that passes through the wall of the paranasal bone (e.g., the ethmoid bone) and that engages soft tissue immediately proximal of such bone. The harder structural material 882 provides better mechanical interaction for anchoring an implant device. However, the softer material and smoother surface of the layer 880 may provide a reduced susceptibility to formation of biofilms thereon relative to the unmodified distal portion of the initial preliminary conduit structure 864. The modified implant device form 860′″ may be used as a final implant device product or may be used as a further preliminary form that is further modified.

Such a modified head form 862″ or modified conduit form 864′ may be further modified, for example, by applying one or more additive materials to one or both of the softer material layers 876 and 880. Such additive materials may include, for example, a wetting agent, an antimicrobial agent and/or other additive materials. FIG. 17 shows the implant device form 860′″ of FIG. 15 after further modification to apply an additive material to each of the layers of softer material 876 and 880. FIG. 17 shows the further modified implant device form 860″″ with shading on a further modified head structure 862′″ and a further modified conduit structure 864″ to show addition of such an additive material to the layer of softer material 876 and the layer of softer material 880. Depositing a wetting agent (e.g., polyvinylpyrrolidone, polyethylene glycol or hyaluronic acid) may further help to prevent biofilm formation on the modified head form 862′″ and the distal portion of the modified conduit form 864″. Such a wetting agent may be deposited from a solution, for example, by dipping or spraying, followed by drying and optionally heating to cure the composition or to improve adhesion of the wetting agent. Addition of an antimicrobial agent may further assist to reduce potential formation of biofilms on the modified head form 862′″ and the distal portion of the modified conduit form 864″. For example, a silver salt may be deposited from the solution applied by dripping or spraying, followed by drying and optionally heating. As another example, silver metal may be deposited by physical vapor deposition or chemical vapor deposition. As another example, an antimicrobial peptide may be deposited from a solution or slurry or may be covalently bonded to exposed polymer (e.g., silicone) of one or both of the softer material layers 876 and 880. As a further example, a polymeric antimicrobial agent (e.g., polyethylene oxide or polyethylene glycol) may be deposited by dip or spray coating from a solution contrary they polymeric antimicrobial agent. In some preferred implementations a wetting agent (e.g., PVP, PEG, hyaluronic acid) and antimicrobial agent (e.g., a polymeric antimicrobial agent and/or an antimicrobial peptide) may be applied together to the same surface.

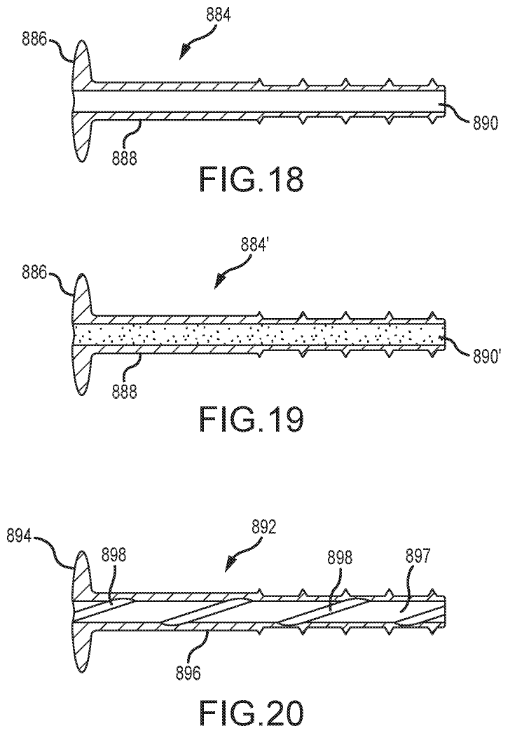

Reference is now made to FIGS. 18 and 19 in relation to an example of modifying a preliminary internal passage structure of a preliminary implant device form to apply a lubricity agent to the internal passage. FIG. 18 shows an initial implant device form 884 including a head 886 and a conduit 888 and having a preliminary internal passage 890 passing through the head 886 and the conduit 888. The preliminary implant device form 884 is illustrated as having features of the implant device of FIG. 8, but it could alternatively be of a different design, for example with one or more features shown in any of FIG. 2-7, 9-17 or 20-28 or 30-35. FIG. 19 shows a modified implant device form 884′ with a modified internal passage structure 890′ following formation of a thin layer of or including a lubricity agent. Such a thin layer of lubricity material may be formed, for example, by casting a film of or including the lubricity agent or precursor therefore onto the walls of the preliminary internal passage 890 and then curing the cast film as necessary to form a final coating with high lubricity. Such a lubricity agent may, for example, be a fluorosilicone polymer or a very smooth silicone film (e.g., similar to layers 876 and 880). One example silicone material for such a smooth film is a film formed by reaction of ethyltriacetoxysilane. As another example, the layers of lubricity material may be deposited by chemical vapor deposition, for example a poly(p-xylylene) polymer (e.g., Parylene N or Parylene C). An internal passage could also be treated to apply an antimicrobial agent, with or without applying a lubricity agent, similar to as described in relation to FIGS. 16-17.

Reference is now made to FIG. 20 showing a cross-section of an implant device 892 having a head 894 and a conduit 896 and with an internal passage 897 extending through the head 894 and the conduit 896. The implant device 892 is illustrated with features of the implant device of FIG. 8, but it could be of a different design, for example including one or more features shown in any of FIG. 2-19 or 21-28. The internal passage 897 of the implant device 892 has a surface geometry including rifling 898 in the form of a spiraling recess extending the along the length of the internal passage 897.

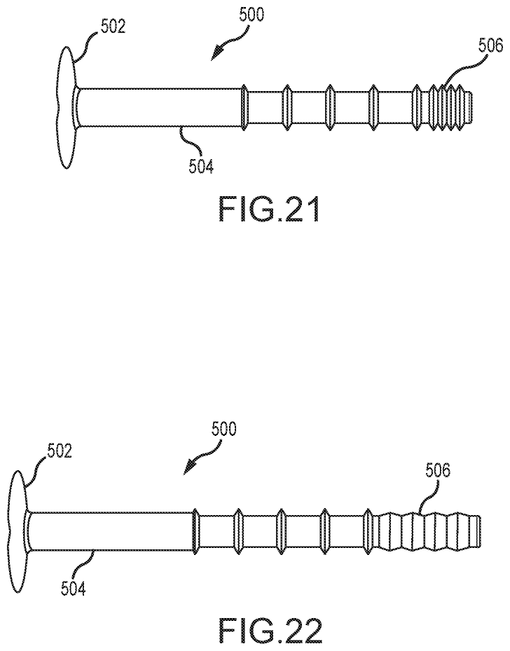

Reference is now made to FIGS. 21 and 22 illustrating one example of an extension portion on a conduit that is extendable and collapsible, or contractible, to lengthen and shorten, respectively, a longitudinal length of a distal portion of a conduit of an implant device. As shown in FIGS. 21 and 22, an implant device 500 includes a head 502 and a conduit 504, and which may have a design similar to that or including any features of an implant device shown in any of FIG. 2-20 or 23-28, except that on a distal portion of the conduit 504, the implant device 500 includes an extension portion 506 of a pleated, or accordion, structure that may be extended and contracted to lengthen and shorten, respectively, a distal portion of the conduit 504 that is to be disposed within the paranasal sinus when the implant device 500 is implanted. The extension portion 506 is shown in a fully contracted, or collapsed, configuration in FIG. 21 and in a fully extended configuration in FIG. 22.

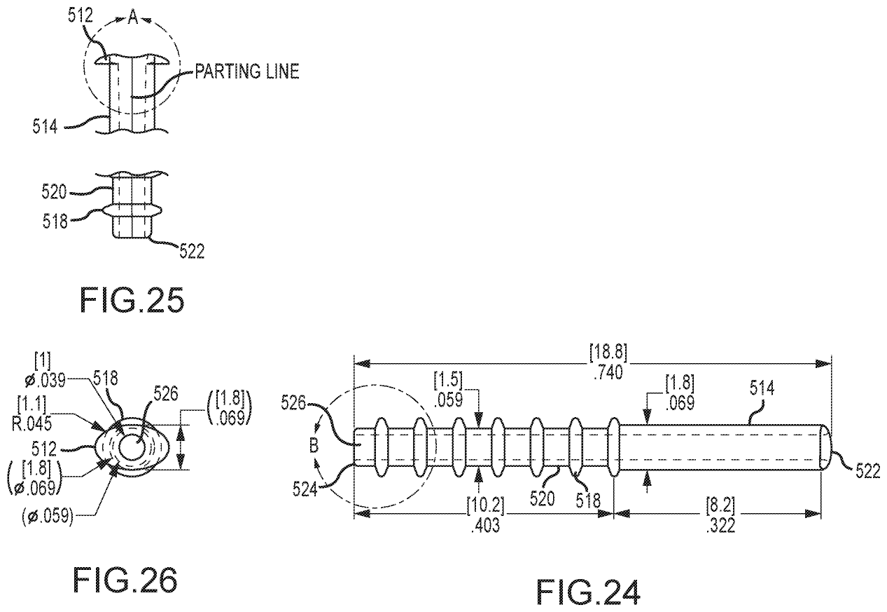

FIGS. 23-28 show another embodiment of a paranasal sinus access implant device. The implant device 510 has a head 512 and a conduit 514 with a first longitudinal portion 516 having a larger minimum wall thickness and a second longitudinal portion 519 having a smaller minimum wall thickness. The implant device 510 has seven anchor protrusions 518 in the form of circumferential ridges and recess areas 520 between pairs of adjacent ridges. The implant device 510 has a proximal end 522 and a distal end 524 and an internal passage 526 that extends from the proximal end 522 through the head 512 and the conduit 514 to the distal end 524. Various details and dimensions for the implant device 510 are shown in FIGS. 24-28. Dimensions are shown in FIGS. 24-28 in inches and in millimeters, with the dimensions in millimeters being in brackets. As will be appreciated, compared to the implant device 800 of FIG. 8, the implant device 510 of FIGS. 23-28 has more circumferential ridges with a closer spacing between the ridges and a smaller head, and with the head 512 having concave surfaces 528 on the distal side of the flanged portions of the head 512 and having convex surfaces 530 on the proximal side of the flanged portions of the head 512 (seen best in FIG. 28). The concave surfaces 528 on each of the flanged portions of the head are in the form of a cup-shaped depression that may trap and hold liquid between soft tissue (e.g., in the orbit) and the concave surfaces 528 to maintain a hydration buffer between the flanged portions of the head 512 and the soft tissue when the implant device is implanted. Other than the head geometry, the implant device 510 of FIGS. 23-28 may have any one or more features shown in or described with respect to any of FIGS. 2-22.

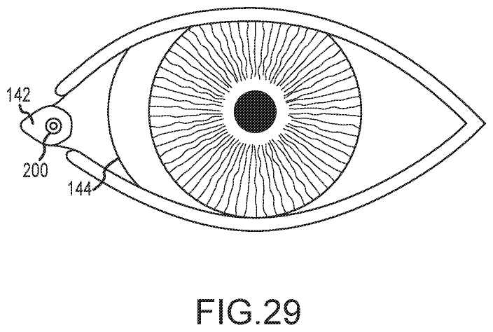

The implant device 510 illustrated in FIG. 23-28 and the implant device 200 illustrated in FIGS. 2-7 with concave undercut head features are particularly advantageous for implantation through the lacrimal caruncle in the lacrimal apparatus in the orbit to connect to a paranasal sinus, for example to the ethmoid sinus. FIG. 29 shows an example of the implant device 200 as implanted through the lacrimal caruncle 142 with the distal side of the flanged portion of the head disposed over tissue of the lacrimal caruncle.

Other than the head geometry, the implant device 200 of FIGS. 2-7 or the implant device 510 of FIGS. 23-28 may have any one or more features shown in or described with respect to any of FIGS. 8-22. For example, in various embodiments, the implant device 200 shown in FIGS. 2-7 or implant device 510 shown in FIGS. 23-28 may include any one or more than one of the following:

a radiopaque feature, with or without a translucent head feature, such as illustrated in and described in relation to FIG. 10;

a softer material layer over a structural material on the head and/or conduit, such as illustrated in and described in relation to any of FIGS. 13-16;

an additive material (e.g., wetting agent or antimicrobial material), such as illustrated in and described in relation to FIG. 17;

a layer of or including a lubricity agent on walls of the internal passage, such as illustrated in and described in relation to FIG. 19;

a rifling geometric feature in the internal passage, such as illustrated in and described in relation to FIG. 20; and

an extension portion on a distal portion of the conduit, such as illustrated in and described in relation to FIGS. 21 and 22.

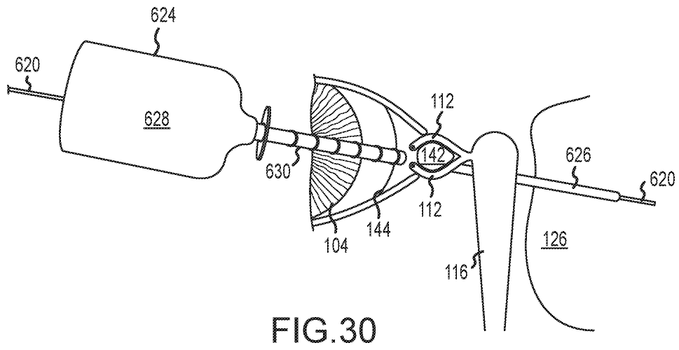

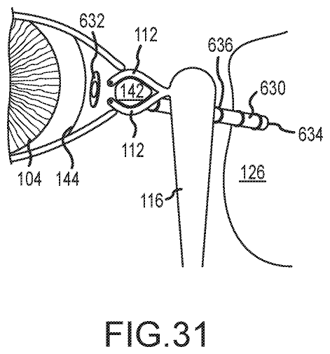

FIGS. 30 and 31 show an example implementation of an implant device 630 to fluidly connect between the lacrimal apparatus in the orbit and the ethmoid sinus 126. To facilitate implantation, the implant device 630 is mounted on a surgical tool, in the form of an implant tool 624. The implant tool may also be referred to as a carrier tool. The implant tool 624 includes a hollow working member 626 and a hand-manipulable handle 628. The implantation tool 624 is shown with the hollow working member 626 advanced through a previously formed fistula between the lacrimal apparatus in the orbit and the paranasal sinus 126. The working member 626 may also be referred to as a carrier member. The implant tool 624 includes an internal passage passing through the handle 628 and the hollow working member 626. As shown in FIG. 30, a guide wire 620 has been threaded through the internal passage of the implant tool 624 to guide the hollow working member 626 to and through the fistula and into the ethmoid sinus 126. The implant device 630 is mounted on the hollow working member 626 of the implant tool 624. FIG. 30 shows the implant tool 624 advanced to a point where the distal end of the implant device 630 is in the vicinity of the proximal end of the fistula opening into the conjunctival sac. From this position, the implant device 630 may be advanced into the fistula with a head of the implant device 630 disposed adjacent the conjunctiva in the conjunctival sac and a distal end of the implant device 630 extending into the ethmoid sinus 626. For example, a surgeon may slide the implant device 630 down the hollow working member 626 for placement through the fistula for implantation or the surgeon may advance the handle 628 to have the handle push the implant device 630 into the fistula for implant placement. The outside diameter of the hollow working member 626 may be sized to closely fit within the inside diameter of the implant device 630 to help prevent the implant device 630 from bunching-up and laterally deforming as the implant device is pushed into the fistula. The handle 628 and the hollow working member 626 form a carrier for the implant device 630. The handle 628 may be retracted and the hollow working member 626 disengaged from the implant device 630 after the implant device has been appropriately positioned for implantation through the fistula. FIG. 31 shows the implant device 630 as implanted and following disengagement of the hollow working member 626 of the implantation tool 624. As implanted, a head 632 at the proximal end of the implant device 630 is located adjacent the conjunctiva in the conjunctival sac within the lacrimal apparatus in the orbit, and the conduit passes through the fistula across tissue including conjunctiva and a wall of the ethmoid bone in which the ethmoid sinus 126 is located. A distal end 634 of the implant device 630 is located in the ethmoid sinus 126. Some anchor protrusions 636 of the conduit of the implant device 630 are disposed within the fistula to engage tissue and help anchor the implant device 630. The implant device 630 may be used to provide access to the ethmoid sinus 126 to perform medical procedures or treatments, for example to administer a treatment composition to the ethmoid sinus or to aspirate fluid from the ethmoid sinus.

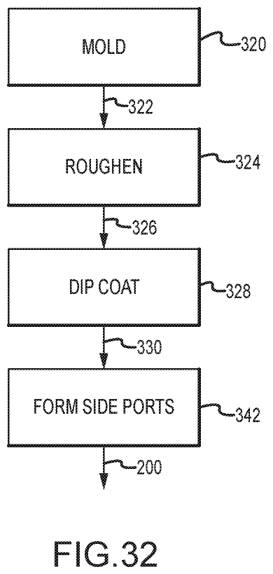

Reference is now made to FIGS. 32-36 to describe example processing to make a paranasal sinus access implant device. The description is exemplified with reference to manufacture of an implant device having the geometry of the implant device 200 shown in FIGS. 2-7, although the description applies generally to manufacture of other implant device embodiments. For example purposes, it is assumed that the implant device will be made of a Shore A durometer 70 silicone material based on polydimethylsiloxane as a primary component of the silicone material. The processing also applies to other polymeric materials, for example to other silicone materials and to other polymers (e.g., polyurethane materials). Some example Shore A 70 durometer silicone materials include Med-4870 (NuSil Technology) and Silpuran 6000/70 (Wacker).

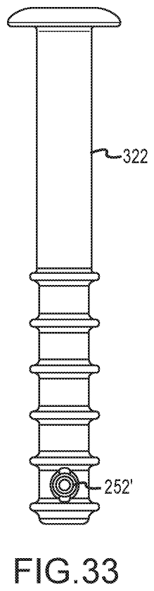

FIG. 32 shows a generalized process block diagram of generalized manufacture processing. As shown in FIG. 32, the generalized processing includes a molding step 320, during which the polymeric precursor resin material is molded, including any curing as desired, to form a preliminary molded form 322 for the implant device. The preliminary molded form generally includes preliminary forms for the conduit, head and internal passage for an implant device, including a preliminary form of a concave undercut on a distal side of a flanged portion of the preliminary head form. The molding includes introducing precursor material in cavity of a mold to form the proper shape for the preliminary molded form 322 and heating and curing the molded form and removing the molded form from the mold following curing. Any appropriate molding technique may be used, for example injection molding, compression molding or transfer molding. One preferred technique is transfer molding. It is also preferred that the mold surface be very highly polished walls to impart a smooth surface to the exterior surfaces and the exposed surfaces within the internal passage of the preliminary molded form. Moreover, the mold should preferably be configured so that no mold partings, and no mold flash, correspond with regions of the internal passage. An example of such a preliminary molded form 322 is shown in FIG. 33. As shown in FIG. 33, the distal portion of the preliminary conduit form where the side ports 250 will be located includes preliminary forms for the circular ridges 252′ where the side ports 250 will be located. However, as prepared during the molding step 320, the preliminary molded form 322 does not include the side ports, which in this example are formed after molding.

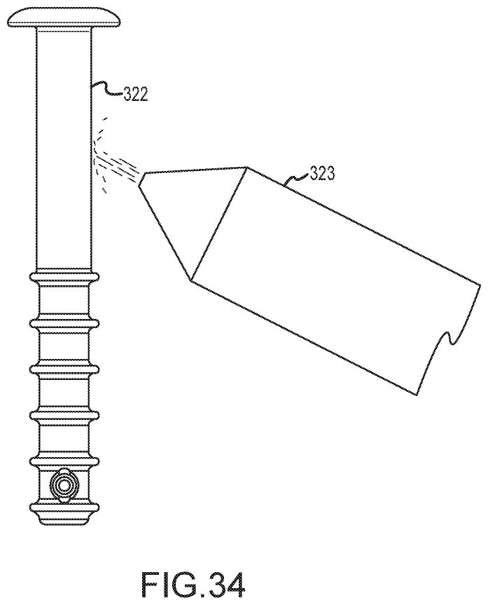

Continuing with reference to FIG. 32, the preliminary molded form 322 is subjected to a roughening step 324 to roughen a portion or all of the exposed exterior surfaces of the preliminary molded form 322, to prepare a preliminary roughened form 326 of the implant device. In some processing variations, only some selected portions of the exterior surfaces in the preliminary molded form 322 will be roughened. The selected portions may be on the head and/or the conduit portions of the preliminary molded form 322. In other processing variations essentially all, of the exposed exterior surfaces of the preliminary molded form 322 (all exterior surfaces of the conduit and the head portions) will be roughened. During the roughening step, the exterior surfaces to be roughened are treated to prepare a roughened exposed surface with a higher average roughness (Ra) (which for convenience may be referred to as a modified average roughness) than an average roughness than the corresponding exposed surface of the preliminary molded form 322 that resulted from the molding step 320 (which for convenience may be referred to as an initial average roughness) Preferably, the roughening does not affect the surface roughness within the internal passage, which remains at the initial average roughness resulting from the molding step 320. Even for exposed surfaces of the preliminary molded form 322 that will be roughened during the roughening step 324, it is still preferred that those surfaces as produced during the molding step 320 are very smooth and have a low average roughness, to provide good uniformity for subsequent processing. In that regard, an initial average surface roughness of exposed surfaces as produced during the molding step 320 may be not larger than 50 nanometers, not larger than 35 nanometers, not larger than 25 nanometers, not larger than 15 nanometers, not larger than 15 nanometers, not larger than 12 nanometers or not larger than 10 nanometers, although such an initial average roughness may be at least 1 nanometer, at least 2 nanometers, at least 5 nanometers, at least 6 nanometers, at least 12 nanometers or even at least 15 nanometers. After the roughening step 324, the modified average roughness on the roughened exterior surfaces of the preliminary roughened form 326 of the implant device may be at least 100 nanometers, at least 150 nanometers, at least 200 nanometers, at least 250 nanometers or at least 300 nanometers, and may often be not larger than 1 micron, not larger than 800 nanometers, not larger than 600 nanometers, not larger than 500 nanometers or not larger than 400 nanometers. Roughening of the surface may provide a variety of advantages. Having a roughened surface on the exterior of the conduit provides a better surface geometry at a micro level for securely anchoring with in-growing tissue when the implant device is implanted, for example in situations when it will be desired to leave the implant device in place for a long time. Also, such a roughened surface, and in particular on the head, provides an advantageous surface for depositing additional material to provide desired finish features to the head, including in relation to size, material and surface properties. The roughening may be accomplished by any suitable technique. One preferred roughening technique is abrasive blasting of surfaces to be treated, with soda blasting using sodium bicarbonate particles being more preferred. FIG. 34 illustrates roughening a surface of the conduit of the preliminary molded form 322 by soda blasting from a blast nozzle 323 to remove material at the blasted surface and form a roughened surface. Such soda blasting preferably uses sodium bicarbonate blasting grains in a micro-size range, for example with an weight average size of 100 microns or smaller or even 50 microns or smaller.

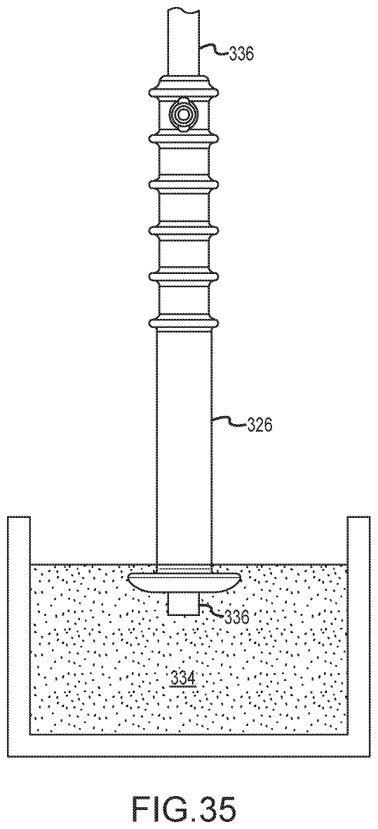

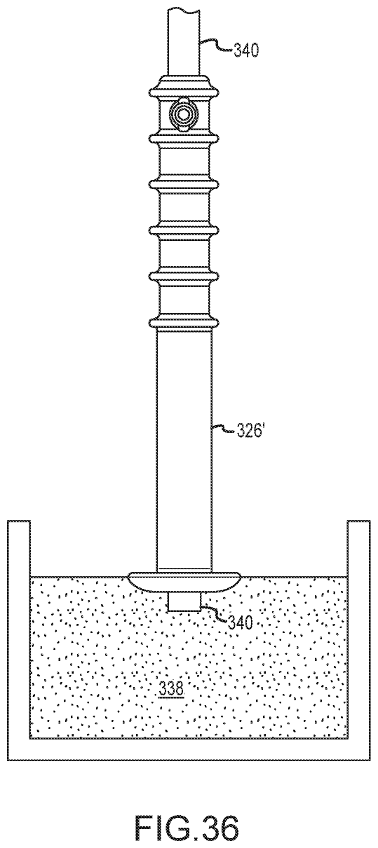

As shown in FIG. 32, the preliminary roughened form 326 is subjected to a dip coating step 328 to form a preliminary coated form 330 of the implant device. In some processing variations, the dip coating step 328 may include selectively coating only selected portions of the preliminary roughened form 326 with coating material to form desired properties at those selected locations or to coat the entire exterior of the roughened preliminary form 326 with coating material to provide desired properties to all exterior surfaces of the implant device. Such coating of the all exterior surfaces may be advantageous, for example, when it is desired to have an extremely smooth surface on all exterior surfaces of the final implant device, for example when it is anticipated that the implant device may be removed after a relatively short time following implantation, in which case promoting good adherence with in-growing tissue may not be desired. In that situation, providing a very smooth surface on the conduit will not promote binding with in-growing surrounding tissue in the same way as a roughened exterior surface on the conduit. In some processing variations, the dip coating 328 will be selective for coating the head to form desired dimensions and surface characteristics for the final head. In some processing variations, the dip coating step 328 may include a single dip-coating stage or may include multiple dip-coating stages. Reference will now be made to FIGS. 35 and 36 to describe one embodiment for a two-stage dip coating operation to selectively coat only surfaces on the head form of the preliminary roughened form 326.

FIG. 35 illustrates an example of a first dip-coating stage in which a proximal portion of the preliminary roughened form 326 is advanced into a first dip-coating formulation 334 to an extent to submerge the head and not to submerge any significant portion of the conduit, even though some small portion of the conduit may be submerged. To prevent contact between any of the exterior of the conduit and the first dip-coating formulation, a protective cover may be disposed around the conduit, if desired. By submerging only the head portion, a coating of coating material is selectively deposited on the head, including on the proximal side of the flanged portion of the head form and on the concave undercut portion on the distal side of the flanged portion of the head form. To avoid filling in the concave undercut portion of the head form, the first dip-coating formulation 334 may have a relatively low concentration of coating material precursor so that the deposited coating layer is relatively thin. After being submerged in the first dip-coating formulation 334 for sufficient time to deposit the desired layer of material, the preliminary roughened form 326 may be withdrawn from the first dip-coating formulation 334. The deposited layer of coating material on the head may be partially or fully cured, such as through application of heat. As shown in FIG. 35, when the head form of the preliminary roughened form 326 is submerged in the first dip-coating formulation 334, a mandrel 336 may be disposed through the internal passage and project beyond the proximal end of the preliminary roughened form 326, preventing coating material from the first dip-coating formulation from coating the walls within the internal passage. When the preliminary roughened form 326 is withdrawn from the first dip-coating formulation 334, the projecting portion of the mandrel 336 may be retracted into the internal passage to pull residual first dip-coating formulation into a proximal portion of the internal passage to coat and provide a smooth transition into the internal passage, for example in the vicinity of the proximal port transition portion of the internal passage. The thickness of the first coating layer resulting from the first dip-coating stage may in some implementations, for example, be on the order of 25 microns. Following any desired curing or partial curing of the first dip-coated layer, the modified preliminary roughened form of the implant device may be subjected to one or more subsequent dip-coating stages. FIG. 36 illustrates the modified preliminary roughened form 326′ that already includes the first dip-coated layer being subjected to a second dip-coating stage during which a proximal portion of the modified preliminary roughened form 326′ is submerged in a second dip-coating formulation 338 to selectively apply a second layer of coating material over a portion of the first dip-coated layer. As shown in FIG. 36, during the second dip-coating stage, the head is submerged to a depth only to cover the proximal side of the flanged portion of the head and not to cover the distal side of the flanged portion of the head, and in particular not to cover the concave undercut. During the second dip-coating stage, a second layer of coating material is deposited on the submerged portion of the head to further build the head to the desired dimensions and to provide the desired surface characteristics. The second dip-coating formulation 338 may include the same material or a different coating material as included in the first dip-coating formulation, for example a different polymeric material or a different durometer of the same type of polymeric material. Likewise, the first dip-coating formulation 334 may deposit the same polymeric material as used to make the preliminary molded form 322 during the molding step 320 or may be a different polymeric material or a different durometer of the same type of polymeric material. In some implementations, one or both of the first dip-coating formulation and second dip-coating formulation may deposit a different durometer silicone (e.g., a softer durometer) over a harder silicone material. In an alternative embodiment, the first dip-coating formulation 334 and/or the second dip-coating formulation 338 may deposit the same type of polymeric material of the same hardness as originally used to form the preliminary form 322 during the molding step 320 (e.g., silicone of same durometer as used during the molding step 320). The second dip-coating formulation 338 may include a much higher concentration than in the first dip-coating formulation (e.g., an order of magnitude or more larger) to deposit a thicker coating layer and without concern for filling in the concave undercut on the distal side of the flanged portion of the head form. For example, in one implementation the layer of coating material deposited during the second dip-coating stage may be on the order of 250 microns. Similar to as shown in FIG. 35, FIG. 36 shows the use of a mandrel 340 inserted through the internal passage to project out of the proximal end of the internal passage to prevent the second dip-coating formulation from entering into and coating walls of the internal passage. When the modified preliminary form 326′ is removed from the second dip-coating formulation, the projecting portion of the mandrel 340 may be retracted into the proximal end of the internal passage to provide some coating of a proximal portion of the internal passage, for example in the vicinity of the proximal port transition portion of the internal passage. After withdrawing the modified preliminary roughened form 326′ from the second dip-coating formulation 338, the coated layer may be cured to a desired extent for the final implant device. Preferably, during curing or partial curing operations following both the first dip-coating stage and the second dip-coating stage, the coated preliminary form of the implant device will be rotated, either individually about the longitudinal axis of the preliminary form or a group of simultaneously processed preliminary forms may be rotated as a unit around a central longitudinal axis for the group. Such rotation helps to provide a more uniform surface finish on the coated surfaces. The exterior surfaces of the coating may be very smooth, and may have an average surface roughness, for example, as described above for the smooth molded surfaces of the preliminary molded form 322, although in some implementations, the smooth surface of the coated material may be even smoother than the molded surfaces. For example, the exposed surfaces of the dip-coated material may have an average roughness that is smaller than the initial average roughness of the exposes surfaces of the preliminary molded form 322. The first dip-coating formulation and/or the second dip-coating formulation 334 or 338 may include any desired liquid vehicle to act as a solvent or suspension medium for coating material precursor components (e.g., silicone material precursors). In some implementations, such a liquid vehicle may be a relatively volatile material for convenient evaporation from deposited material (e.g., xylene).

A contemplated alternative implementation to the dip coat step 328 is to use a different deposition teaching to apply the contemplated different deposited layers of coating material for one or more material deposition operation during the dip coat step 328, for example by spray coating. For example, one or both of the material application operations illustrated in FIGS. 35 and 36 could be performed by spray coating, or another material deposition technique, rather than dip coating.

As shown in FIG. 32, the preliminary coated form 330 resulting from the dip-coating step 328 is subjected to a step 342 to form the side ports. The step 342 may include a punching operation where the ports 252 for the final implant device are punched using a punching die. A result of the step 342 is the final form for the implant device 200. This “final” form of the implant device 200 resulting from the general processing of FIG. 32 may be a final product form for packaging and use as a final implant device for implantation to provide access from a lacrimal apparatus to a paranasal sinus, or may be further processed or modified to prepare such a final product form. Also, the preliminary roughened form 326 or the preliminary coated form 330 may be used as such a final product form or may be used as a preliminary form for processing other than the subsequent processing shown in FIG. 32.

A variety of medical treatments and procedures may be performed through a paranasal sinus access implant device implanted to provide access to a paranasal sinus. Fluid treatment compositions may be administered to a paranasal sinus through the implant device. Fluid may be aspirated from a paranasal sinus through the implant device. One or more medical devices may be inserted into the paranasal sinus through the implant device.

Some example implementation combinations, and for various types of implementation applications, which may be the subject of claims with or without additional features as disclosed above, are disclosed as follows:

1. A paranasal sinus access implant device useful for implantation in a human to fluidly connect a lacrimal apparatus to a paranasal sinus through a fistula formed between the lacrimal apparatus and the paranasal sinus, the implant device comprising:

a proximal end at a first longitudinal end of the device to be disposed in the lacrimal apparatus when the device is implanted and a distal end at a second longitudinal end of the device to be disposed in the paranasal sinus when the device is implanted;

a length longitudinally along the implant device between the proximal end and the distal end in a range of from 8 millimeters to 50 millimeters;

a conduit, located between the proximal end and distal end, to be disposed through a fistula between the lacrimal apparatus and the paranasal sinus when the device is implanted, the conduit comprising a distal portion to be disposed in the paranasal sinus when the implant device is implanted;

a head adjacent the proximal end and connected with the conduit, the head including a flanged portion to be disposed in the lacrimal apparatus when the device is implanted;

an internal passage through the head and the conduit, to provide a fluid communication path between the lacrimal apparatus and the paranasal sinus when the device is implanted, the internal passage having a width through at least a portion of the conduit in a range of from 0.5 millimeter to 2.5 millimeters; and

further comprising any one or combination of more than one of the following features:

(a) an exposed surface of exposed material and having an average roughness (Ra) of not larger than 50 nanometers, wherein the exposed surface is exposed in the internal passage;

(b) an exposed surface having an average roughness of not larger than 50 nanometers, wherein the exposed surface is a first exposed surface and the average roughness is a first average roughness, and the implant device includes a second exposed surface having a second average roughness of at least 100 nanometers;

(c) an exposed surface of exposed material and having an average roughness of not larger than 50 nanometers, wherein the exposed surface extends over essentially all exterior of the implant device;

(d) the head has a perimeter transverse to a longitudinal axis of the implant device and the flanged portion of the head includes a concave undercut portion on a distal side of the flanged portion, with the concave undercut extending 360 degrees around the axis within the perimeter of the head;

(e) the internal passage includes a proximal port transition portion including a tapering cross-section that tapers in a direction from the proximal end toward the distal end of the implant device; and

(f) the internal passage includes a distal port transition portion with tapering cross-section that tapers in a direction from the distal end toward the proximal end of the implant device.

2. An implant device according to example implementation combination 1, comprising feature (a), and wherein:

the average roughness of the exposed surface in the internal passage is in a range having a lower limit of 1 nanometer, 2 nanometers, 5 nanometers, 6 nanometers, 10 nanometers, and 12 nanometers, 15 nanometers and 20 nanometers and an upper limit of 50 nanometers, 35 nanometers, 25 nanometers, 20 nanometers, 15 nanometers, 12 nanometers and 10 nanometers, with the upper limit being larger than the lower limit.

3. An implant device according to example implementation combination 2, wherein the exposed surface includes essentially all surface exposed in the internal passage.

4. An implant device according to either one of example implementation combination 2 or example implementation combination 3, wherein the internal passage includes the feature (e).

5. An implant device according to any one of example implementation combinations 2-4, wherein the internal passage includes the feature (f).

6. An implant device according to any one of example implementation combinations 2-5, wherein the exposed surface is a second exposed surface and the average roughness is a second average roughness and the implant device comprises a first exposed surface including exterior surface of the head and having first average roughness, and

the second exposed surface has a second average roughness that is larger than the first average roughness by at least 5 nanometers, at least 7 nanometers or at least 10 nanometers, and optionally is not more than 35 nanometers, not more than 25 nanometers, or not more than 15 nanometers larger than the first average roughness.

7. An implant device according to example implementation combination 6, wherein the first average roughness is in a range having a lower limit of 1 nanometer, 2 nanometers or 5 nanometers and an upper limit of 40 nanometers, 35 nanometers, 25 nanometers, 20 nanometers, 15 nanometers, 12 nanometers or 10 nanometers.

8. An implant device according to any one of example implementation combinations 1-5, including feature (b) and wherein:

the second exposed surface includes an exposed exterior surface of the conduit;

the first average roughness is in a range having a lower limit of 1 nanometer, 2 nanometers, 5 nanometers or 10 nanometers and an upper limit of 50 nanometers, 40 nanometers, 35 nanometers, 25 nanometers, 20 nanometers, 15 nanometers, 12 nanometers or 10 nanometers; and

the second average roughness is in a range having a lower limit of 100 nanometers, 150 nanometers, 200 nanometers, 250 nanometers or 300 nanometers and an upper limit of 1 micron, 800 nanometers, 600 nanometers, 500 nanometers or 400 nanometers.

9. An implant device according to example implementation combination 8, wherein the second exposed surface includes essentially all exposed exterior surface of the conduit.

10. An implant device according to either one of example implementation combination 8 or example implementation combination 9, wherein the first exposed surface includes an exposed exterior surface of the head.

11. An implant device according to Claim 10, wherein the head includes a proximal side disposed away from the distal end of the implant device and the first exposed surface includes exterior surface of the proximal side of the head.

12. An implant device according to either one of example implementation combination 10 or example implementation combination 11, wherein the first exposed surface includes essentially all exposed exterior surface of the head.

13. An implant device according to any one of example implementation combinations 10-12, comprising a third exposed surface including exposed surface in the internal passage, and wherein the third exposed surface has a third average roughness that is larger than the first average roughness and smaller than the second average roughness.

14. An implant device according to example implementation combination 13, wherein:

the third average roughness is at least 50 nanometers smaller, at least 100 nanometers smaller, at least 150 nanometers smaller or at least 200 nanometers smaller than the second average roughness; and

the third average roughness is at least 5 nanometers larger, at least 7 nanometers larger, at least 10 nanometers larger or at least 15 nanometers larger than the first average roughness.

15. An implant device according to example implementation combination 14, wherein:
the first average roughness is in a range having a lower limit of 1 nanometer, 2 nanometers, 5 nanometers or 10 nanometers and an upper limit of 40 nanometers, 35 nanometers, 25 nanometers, 20 nanometers, 15 nanometers, 12 nanometers or 10 nanometers;

the second average roughness is in a range having a lower limit of 100 nanometers, 150 nanometers, 200 nanometers, 250 nanometers or 300 nanometers and an upper limit of 1 micron, 800 nanometers, 600 nanometers, 500 nanometers or 400 nanometers; and

and the third average roughness is in a range having a lower limit of 1 nanometer, 2 nanometers, 5 nanometers, 6 nanometers, 10 nanometers, and 12 nanometers, 15 nanometers and 20 nanometers and an upper limit of 50 nanometers, 35 nanometers, 25 nanometers, 20 nanometers, 15 nanometers, 12 nanometers and 10 nanometers and, with the upper limit being larger than the lower limit.

16. An implant device according to any one of example implementation combinations 8-15, wherein the first exposed surface includes exposed surface in the internal passage.

17. An implant device according to example implementation combination 16, wherein the first exposed surface includes essentially all exposed surface in the internal passage.

18. An implant device according to any one of example implementation combinations 8-17, wherein the internal passage is free of mold flash.

19. An implant device according to any one of example implementation combinations 8-18, wherein:

an exterior of the conduit comprises an anchoring surface feature including protrusion areas and recess areas, with the protrusion areas having a height relative to the recess areas in a range having a lower limit of 0.1 millimeter, 0.2 millimeter, 0.25 millimeter or 0.3 millimeter and an upper limit of 2 millimeters, 1.5 millimeter, 1 millimeter, 0.75 millimeter, 0.5 millimeter, 0.45 millimeter or 0.4 millimeter, and

the second exposed surface includes exposed surfaces of the protrusion areas and recess areas.

20. An implant device according to any one of example implementation combinations 8-19, wherein the first exposed surface and the second exposed surface each has an area of at least 1 square millimeter, at least 2 square millimeters, at least 3 square millimeters, or at least 5 square millimeter or at least 10 square millimeters.

21. An implant device according to any one of example implementation combinations 8-20, wherein the flanged portion of the head includes a distal side disposed toward the distal end of the implant device and the first exposed surface includes exterior surface of the distal side of the head.

22. An implant device according to example implementation combination 21 wherein the distal side of the flanged portion has a concave undercut, and the first exposed surface includes an exposed exterior surface of the concave undercut.

23. An implant device according to example implementation combination 22, wherein:

the head has an aerial perimeter transverse to a longitudinal axis of the implant device and the flanged portion and the concave undercut extend for 360 degrees about the axis; and

the concave undercut has a depth relative to a distal lip of the flanged portion in a range having a lower limit of 0.05 millimeter, 0.07 millimeter, 0.1 millimeter, 0.13 millimeter, 0.15 millimeter and 0.17 millimeter and an upper limit of 0.6 millimeter, 0.5 millimeter, 0.4 millimeter, 0.3 millimeter, 0.25 millimeter, 0.23 millimeter and 0.2 millimeter.

24. An implant device according to example implementation combination 23, wherein the aerial perimeter of the head is circular and the circle of the perimeter of the head has a diameter in a range having a lower limit of 2.75 millimeters, 3 millimeters, 3.25 millimeters or 3.5 millimeters and an upper limit of 6 millimeters, 5.5 millimeters, 5 millimeters, 4.75 millimeters or 4.5 millimeters.

25. An implant device according to example implementation combination 24, wherein the concave undercut has a depth relative to a distal lip of the flanged portion in a range of from 0.1 millimeter to 0.6 millimeter.

26. An implant device according to example implementation combination 22, wherein:

the head has an aerial perimeter transverse to a longitudinal axis of the implant device and the head includes at least two opposing said flanged portions extending toward the perimeter on opposing sides of the axis, and wherein each said flanged portion includes a said concave undercut; and

each said concave undercut has a depth relative to a distal lip of the flanged portion in a range having a lower limit of 0.05 millimeter, 0.07 millimeter, 0.1 millimeter, 0.13 millimeter, 0.15 millimeter and 0.17 millimeter and an upper limit of 0.6 millimeter, 0.5 millimeter, 0.4 millimeter, 0.3 millimeter, 0.25 millimeter, 0.23 millimeter and 0.2 millimeter.

27. An implant device according to example implementation combination 1, comprising feature (c).

28. An implant device according to example implementation combination 27, comprising feature (a).

29. An implant device according to example implementation combination 27, wherein the exposed surface is a first exposed surface and the average roughness is a first average roughness and the implant device comprises a second exposed surface exposed in the internal passage having a second average roughness of not larger than 50 nanometers and that is larger than the first average roughness by at least 5 nanometers, at least 7 nanometers or at least 10 nanometers.

30. An implant device according to example implementation combination 29, wherein:

the first average roughness is in a range having a lower limit of 1 nanometer, 2 nanometers or 5 nanometers and an upper limit of 40 nanometers, 35 nanometers, 25 nanometers, 20 nanometers, 15 nanometers, 12 nanometers or 10 nanometers; and

the second average roughness is in a range having a lower limit of 6 nanometers, 10 nanometers, and 12 nanometers, 15 nanometers and 20 nanometers and an upper limit of 50 nanometers, 35 nanometers, 25 nanometers, 20 nanometers, 15 nanometers, 12 nanometers and 10 nanometers, with the upper limit being larger than the lower limit.

31. An implant device according to example implementation combination 27, wherein the exposed surface includes surface exposed in the internal passage.

32. An implant device according to example implementation combination 31, wherein the exposed surface includes essentially all surface exposed in the internal passage.

33. An implant device according to any one of example implementation combinations 1-21 and 27-32, comprising feature (d).

34. An implant device according to example implementation combination 33, wherein the concave undercut has an average roughness a range having a lower limit of 1 nanometer, 2 nanometers or 5 nanometers and an upper limit of 50 nanometers, 40 nanometers, 35 nanometers, 25 nanometers, 20 nanometers, 15 nanometers, 12 nanometers or 10 nanometers.

35. An implant device according to either one of example implementation combination 33 or example implementation combination 34, wherein the head has an aerial perimeter transverse to a longitudinal axis of the implant device and the flanged portion and the concave undercut extend for 360 degrees about the axis.

36. An implant device according to example implementation combination 35, wherein the aerial perimeter of the head is circular.

37. An implant device according to example implementation combination 36 wherein the circle of the perimeter of the head has a diameter in a range having a lower limit of 2.75 millimeters, 3 millimeters, 3.25 millimeters or 3.5 millimeters and an upper limit of 6 millimeters, 5.5 millimeters, 5 millimeters, 4.75 millimeters or 4.5 millimeters.

38. An implant device according to any one of example implementation combinations 35-37, wherein the concave undercut has a uniform cross-section in a plane including the axis at all radial locations about the axis.

39. An implant device according to any one of example implementation combinations 21-26 and 33-38, wherein the concave undercut has a radius of curvature in a range having a lower limit of 0.1 millimeter or 0.2 millimeter and an upper limit of 0.7 millimeter, 0.6 millimeter or 0.5 millimeter.

40. An implant device according to any one of claims 33-39, wherein the concave undercut has a depth relative to a distal lip of the flanged portion in a range having a lower limit of 0.05 millimeter, 0.07 millimeter, 0.1 millimeter, 0.13 millimeter, 0.15 millimeter and 0.17 millimeter and an upper limit of 0.6 millimeter, 0.5 millimeter, 0.4 millimeter, 0.3 millimeter, 0.25 millimeter, 0.23 millimeter and 0.2 millimeter.

41. An implant device according to any one of example implementation combinations 1-40, wherein the head has a depth dimension in a direction from the proximal end to the distal end of the implant device in a range having a lower limit of 0.4 millimeter, 0.45 millimeter, 0.5 millimeter or 0.55 millimeter and an upper limit of 1.25 millimeters, 1 millimeter or 0.75 millimeter.

42. An implant device according to any one of example implementation combinations 1-41, comprising feature (e).

43. An implant device according to Claim 42, wherein the proximal port transition portion is in the head.

44. An implant device according to either one of example implementation combination 42 or example implementation combination 43, wherein at all locations of the proximal port transition portion in a longitudinal direction from the proximal end to the distal end of the implant device, the proximal port transition portion has a circular cross-section normal to a longitudinal axis of the implant device.

45. An implant device according to example implementation combination 44, wherein the diameter of the circular cross-section of the internal passage tapers by an amount in a range having a lower limit of at least 0.1 millimeter, 0.2 millimeter, 0.3 millimeter, 0.4 millimeter or 0.5 millimeter from an initial diameter at a proximal end of the proximal port transition portion in a range having a lower limit of 0.75 millimeter, 1 millimeter, 1.5 millimeters or 2 millimeters and an upper limit of 4 millimeters, 3.5 millimeters, 3 millimeters, or 2.5 millimeters or 2 millimeters (with the upper limit being larger than the lower limit) to a tapered diameter at a distal end of the proximal port transition portion in a range having a lower limit of 0.4 millimeter, 0.5 millimeter, 0.6 millimeter, 0.75 millimeter or 1 millimeter and an upper limit of 2.5 millimeters, 2.25 millimeters, 2 millimeters, 1.75 millimeters, 1.5 millimeters and 1.25 millimeters.

46. An implant device according to any one of example implementation combinations 43-45, wherein the proximal port transition portion has a length from a proximal end to a distal end of the proximal port transition portion in a range of from 0.4 millimeter, 0.45 millimeter, 0.5 millimeter or 0.55 millimeter and an upper limit of 2 millimeters, 1.5 millimeters, 1.25 millimeters, 1 millimeter or 0.75 millimeter.

47. An implant device according to any one of example implementation combination 43-45, comprising feature (f), and wherein the internal passage has an intermediate section extending longitudinally from a distal end of the proximal port transition portion to a proximal end of the distal port transition portion and the intermediate portion has a constant width along the length of the intermediate portion.

48. An implant device according to example implementation combination 47, wherein the intermediate portion of the internal passage has a constant circular cross-section along the length of the intermediate portion, wherein the constant circular cross-section has a diameter in a range having a lower limit of 0.4 millimeter, 0.5 millimeter, 0.6 millimeter, 0.75 millimeter or 1 millimeter and an upper limit of 2.5 millimeters, 2.25 millimeters, 2 millimeters, 1.75 millimeters, 1.5 millimeters and 1.25 millimeters.

49. An implant device according to any one of example implementation combinations 1-48, comprising feature (f).

50. An implant device according to Claim 49, wherein the distal port transition portion is in a distal portion of the conduit.

51. An implant device according to either one of example implementation combination 49 or example implementation combination 50, wherein at all locations of the distal port transition portion in a longitudinal direction from the proximal end to the distal end of the implant device, the distal port transition portion has a circular cross-section normal to a longitudinal axis of the implant device.

52. An implant device according to example implementation combination 51, wherein the diameter of the circular cross-section of the internal passage in the distal port transition portion tapers by an amount in a range having a lower limit of 0.1 millimeter, 0.15 millimeter, 0.25 millimeter, 0.35 millimeter or 0.5 millimeter and an upper limit of 2 millimeters, 1.5 millimeters, 1.25 millimeters or 1 millimeter from a distal end of the distal port transition portion to a proximal end of the distal port transition portion, and the proximal end of the distal transition portion has a circular cross-section with a diameter in a range having a lower limit of 0.4 millimeter, 0.5 millimeter, 0.6 millimeter, 0.75 millimeter or 1 millimeter and an upper limit of 2.5 millimeters, 2.25 millimeters, 2 millimeters, 1.75 millimeters, 1.5 millimeters and 1.25 millimeter.

53. An implant device according to any one of example implementation combinations 49-52, wherein the distal port transition portion has a length from a proximal end to a distal end of the distal port transition portion in a range having a lower limit of 0.25 millimeter, 0.3 millimeter, 0.4 millimeter, 0.5 millimeter, 0.75 millimeter or 1 millimeter and an upper limit of 3 millimeters, 2.5 millimeters, 2 millimeters, 1.5 millimeters or 1.25 millimeters.

54. An implant device according to any one of example implementation combinations 49-53, wherein an exterior of the conduit comprises an anchoring surface feature including protrusion areas and recess areas, with the protrusion areas having a height relative to the recess areas in a range having a lower limit of 0.1 millimeter, 0.2 millimeter, 0.25 millimeter or 0.3 millimeter and an upper limit of 2 millimeters, 1.5 millimeter, 1 millimeter, 0.75 millimeter, 0.5 millimeter, 0.45 millimeter or 0.4 millimeter, and a proximal end of the distal port transition portion is located no closer to the proximal end of implant device than a said protrusion area that is farthest from the proximal end of the implant device.

55. An implant device according to any one of example implementation combinations 1-54, wherein:

the length of the implant device is in a range having a lower limit of 8 millimeters, 10 millimeters, 12 millimeters or 15 millimeters and an upper limit of 50 millimeters, 40 millimeters, 30 millimeters, 25 millimeters or 20 millimeters

an exterior of the conduit comprises an anchoring surface feature including protrusion areas and recess areas, with the protrusion areas including protrusion occurrences in the form of circumferential ridges each having a circular perimeter normal to the longitudinal axis of the implant device and spaced along the conduit and separated by the recess areas, wherein the anchoring surface feature includes at least 5 of the circumferential ridges, and optionally not more than 8 of the circumferential ridges.

56. An implant device according to example implementation combination 55, wherein the circular perimeter of each said circumferential ridge has a diameter of in a range of from 2.3 millimeters to 2.6 millimeters.

57. An implant device according to either one of example implementation combination 55 or example implementation combination 55, wherein the protrusion occurrences have a height relative to the recess areas in a range of from 0.25 millimeter to 0.5 millimeter;

58. An implant device according to any one of example implementation combinations 55-57, wherein the circumferential ridges have a center-to-center spacing in a range of from 1.2 millimeter to 1.9 millimeter.

59. An implant device according to any one of example implementation combinations 55-58, wherein the protrusion circumferential ridges have a base toward the internal passage and a top opposite the base and away from the internal passage and a spacing between adjacent said bases of adjacent said circumferential ridges is in a range of from 0.9 millimeter to 1.5 millimeters.

60. An implant device according to any one of example implementation combinations 55-59, wherein a nearest said circumferential ridge to the proximal end of the implant device is spaced a distance in a range of from 7 to 11 millimeters from the proximal end of the implant device.

61. An implant device according to any one of example implementation combinations 55-60, wherein between a proximal end of the conduit and a nearest said circumferential ridge to the proximal end of the implant device, the conduit has a uniform circular perimeter normal to the longitudinal axis of the implant device having a first diameter in a range of from 1.9 millimeters to 2.1 millimeters.

62. An implant device according to any one of example implementation combinations 55-61, wherein at the recess areas the conduit has a circular perimeter normal to the longitudinal axis of the implant device having a second diameter that is smaller than the first diameter and that is in a range of from 1.6 millimeters to 1.9 millimeters.

63. An implant device according to any one of example implementation combinations 55-62, comprising at least one side port through a wall of the conduit into the internal passage, wherein the side port is located between two adjacent ones of the circumferential ridges and the side port is encircled by a raised ridge on the exterior of the conduit between the two said adjacent circumferential ridges, the raised circular ridge that is raised relative to the recess area between the two said adjacent circumferential ridges.

64. An implant device according to example implementation combination 63, wherein the raised circular ridge is raised relative to the adjacent recessed area by an amount in a range having a lower limit of 0.01 millimeter, 0.025 millimeter and 0.05 millimeter and an upper limit of 0.25 millimeter, 0.2 millimeter, 0.15 millimeter, 0.1 millimeter and 0.075 millimeter.

65. An implant device according to either one of example implementation combination 63 or example implementation combination 64, wherein the side port has a circular cross section with a diameter in a range having a lower limit of 0.3 millimeter, 0.4 millimeter or 0.5 millimeter and an upper limit of 1 millimeter, 0.9 millimeter, 0.8 millimeter or 0.7 millimeter.

66. An implant device according to any one of example implementation combinations 63-65, wherein the side port is located within 5 millimeters, within 4 millimeters, or within 3 millimeters of the distal end of the implant device.

67. An implant device according to any one of example implementation combinations 63-66, wherein the two adjacent said circumferential ridges are a closest adjacent pair of said circumferential ridges to the distal end of the implant device.

68. An implant device according to any one of example implementation combinations 63-67, comprising 2 said side ports between the adjacent two said circumferential ridges on opposing sides of the conduit.

69. A paranasal sinus access implant device useful for implantation in a human to fluidly connect a lacrimal apparatus to a paranasal sinus through a fistula formed between the lacrimal apparatus and the paranasal sinus, the implant device comprising:

a proximal end at a first longitudinal end of the device to be disposed in the lacrimal apparatus when the device is implanted and a distal end at a second longitudinal end of the device to be disposed in the paranasal sinus when the device is implanted;

a length longitudinally along the implant device between the proximal end and the distal end in a range having a lower limit of 8 millimeters, 10 millimeters, 12 millimeters or 15 millimeters and an upper limit of 50 millimeters, 40 millimeters, 30 millimeters, 25 millimeters or 20 millimeters;

a conduit, located between the proximal end and distal end, to be disposed through a fistula between the lacrimal apparatus and the paranasal sinus when the device is implanted, the conduit comprising a distal portion to be disposed in the paranasal sinus when the implant device is implanted;

a head adjacent the proximal end and connected with the conduit, the head including a flanged portion to be disposed in the lacrimal apparatus when the device is implanted;

an internal passage through the head and the conduit, to provide a fluid communication path between the lacrimal apparatus and the paranasal sinus when the device is implanted, the internal passage having a width through at least a portion of the conduit in a range having a lower limit of 0.5 millimeter, 0.6 millimeter, 0.75 millimeter or 1 millimeter and an upper limit of 2.5 millimeters, 2.25 millimeters, 2 millimeters, 1.75 millimeters, 1.5 millimeters and 1.25 millimeters;

the head having a circular perimeter normal to the longitudinal axis of the implant device with a diameter in a range having a lower limit of 2.75 millimeters, 3 millimeters, 3.25 millimeters or 3.5 millimeters and an upper limit of 6 millimeters, 5.5 millimeters, 5 millimeters, 4.75 millimeters or 4.5 millimeters;

an exterior of the conduit comprising an anchoring surface feature including protrusion areas and recess areas, with the protrusion areas including protrusion occurrences in the form of circumferential ridges each having a circular perimeter normal to the longitudinal axis of the implant device with a diameter of in a range of from 2.3 millimeters to 2.6 millimeters and spaced along the conduit and separated by the recess areas, wherein the anchoring surface feature includes at least 5 of the circumferential ridges, and optionally not more than 8 of the circumferential ridges;

the circumferential ridges having a height relative to the recess areas in a range having a lower limit of 0.2 millimeter, 0.25 millimeter or 0.3 millimeter and an upper limit of 0.5 millimeter or 0.4 millimeter;

the circumferential ridges having a center-to-center spacing in a range having a lower limit of 1.2 millimeter, 1.3 millimeter, 1.4 millimeter or 1.5 millimeter and an upper limit of 1.9 millimeter, 1.8 millimeter or 1.7 millimeter;

the circumferential ridges having a base toward the internal passage and a top opposite the base and away from the internal passage and a spacing between adjacent said bases of adjacent said circumferential ridges being in a range having a lower limit of 0.9 millimeter, 1 millimeter or 1.1 millimeter and an upper limit of 1.5 millimeters, 1.4 millimeters or 1.3 millimeters;

a nearest said circumferential ridge to the proximal end of the implant device being spaced a distance in a range having a lower limit of 7 millimeters, 7.5 millimeters or 8 millimeters and an upper limit of 11 millimeters, 10 millimeters and 9 millimeters from the proximal end of the implant device;

between a proximal end of the conduit and the said nearest circumferential ridge to the proximal end of the implant device, the conduit having a uniform circular perimeter normal to the longitudinal axis of the implant device having a first diameter in a range of from 1.9 millimeters to 2.1 millimeters; and

at the recess areas the conduit having a circular perimeter normal to the longitudinal axis of the implant device having a second diameter that is smaller than the first diameter and that is in a range of from 1.6 millimeters to 1.9 millimeters.

70. An implant device according to example implementation combination 69, comprising at least one side port through a wall of the conduit into the internal passage, wherein the side port is located between two adjacent ones of the circumferential ridges and the side port is encircled by a raised ridge on the exterior of the conduit between the two said adjacent circumferential ridges, the raised circular ridge that is raised relative to the recess area between the two said adjacent circumferential ridges.

71. An implant device according to example implementation combination 70, wherein the raised circular ridge is raised relative to the adjacent recessed area by an amount in a range having a lower limit of 0.01 millimeter, 0.025 millimeter and 0.05 millimeter and an upper limit of 0.25 millimeter, 0.2 millimeter, 0.15 millimeter, 0.1 millimeter and 0.075 millimeter.

72. An implant device according to either one of example implementation combination 70 or example implementation combination 71, wherein the side port has a circular cross section with a diameter in a range having a lower limit of 0.3 millimeter, 0.4 millimeter or 0.5 millimeter and an upper limit of 1 millimeter, 0.9 millimeter, 0.8 millimeter or 0.7 millimeter.

73. An implant device according to any one of example implementation combinations 70-72, wherein the side port is located within 5 millimeters, within 4 millimeters or within 3 millimeters of the distal end of the implant device.

74. An implant device according to any one of example implementation combinations 70-73, wherein the two adjacent said circumferential ridges are a closest adjacent pair of said circumferential ridges to the distal end of the implant device.

75. A method for making a paranasal access implant device constructed of polymeric material, wherein the paranasal access implant device comprises:

a proximal end at a first longitudinal end of the device to be disposed in the lacrimal apparatus when the device is implanted and a distal end at a second longitudinal end of the device to be disposed in the paranasal sinus when the device is implanted;

a length longitudinally along the implant device between the proximal end and the distal end in a range of from 8 millimeters to 50 millimeters;

a conduit, located between the proximal end and distal end, to be disposed through a fistula between the lacrimal apparatus and the paranasal sinus when the device is implanted, the conduit comprising a distal portion to be disposed in the paranasal sinus when the implant device is implanted;

a head adjacent the proximal end and connected with the conduit, the head including a flanged portion to be disposed in the lacrimal apparatus when the device is implanted;

an internal passage through the head and the conduit, to provide a fluid communication path between the lacrimal apparatus and the paranasal sinus when the device is implanted, the internal passage having a width through at least a portion of the conduit in a range of from 0.5 millimeter to 2.5 millimeters;

the method comprising:

preparing a preliminary form of the implant device including preliminary forms of the head, conduit and internal passage; and

modifying the preliminary form, wherein the modifying comprises treating an exposed surface on an exterior of the preliminary form to prepare a roughened exposed surface having a higher average roughness than the exposed surface prior to the treating.

76. A method according to example implementation combination 75, wherein the preliminary form is a molded article and the exterior surface prior to the treating is as resulting from a molding process to prepare the preliminary form.

77. A method according to either one of example implementation combination 75 or example implementation combination 76, wherein the average roughness of the exposed surface prior to the treating is no larger than 50 nanometers.

78. A method according to any one of example implementation combinations 75-77, wherein the average roughness of the roughened exposed surface is at least 100 nanometers.

79. A method according to any one of example implementation combinations 75-78, wherein the treating comprises removing material from the preliminary form at the exposed surface.

80. A method according to example implementation combination 79, wherein the removing material comprises abrasive blasting of the exposed surface with particulate abrasive material.

81. A method according to example implementation combination 79, wherein the treating comprises soda blasting the exposed surface with particulate sodium bicarbonate.

82. A method according to any one of example implementation combinations 75-81, wherein the exposed surface includes essentially all surfaces exposed on the exterior of the preliminary form and the roughened surface includes essentially all surfaces exposed on the exterior of a modified preliminary form following the treating.

83. A method according to any one of example implementation combinations 75-82, wherein the treating does not roughen exposed surfaces in the internal channel located more than 2 millimeters into the internal passage from a proximal end of the internal passage.

84. A method according to any one of example implementation combinations 75-83, wherein the treating does not roughen essentially any portion of exposed surface in the internal channel adjacent a distal end of the internal passage.

85. A method according to any one of example implementation combinations 75-84, wherein the treating is a first treating and the method comprises second treating a modified form of the implant device including the roughened exterior surface resulting from the first treating, the second treating comprising:

forming a smoothed exposed surface over at least a portion of the area of the roughened exposed surface, the smoothed exposed surface having an average roughness smaller than the average roughness of the roughened exposed surface and smaller than the average roughness of the exposed surface prior to the first treating.

86. A method according to example implementation combination 85, wherein the smoothed exposed surface has an average surface roughness in a range of from 1 nanometer to 25 nanometers.

87. A method according to either one of example implementation combination 85 or example implementation combination 86, wherein the second treating is performed to selectively form the smoothed exposed surfaces over a first portion of the roughened exposed surface and not over a second portion of the roughened exposed surface, which second portion of the roughened exposed surface remains following the second treating.

88. A method according to example implementation combination 87, wherein:

the first portion of the exposed roughened surface and the smoothed exterior surface are on the head;

and the second portion of the exposed roughened surface is on the conduit.

89. A method according to example implementation combination 88, wherein a second modified form of the implant device resulting from the second treating comprises the smoothed exposed surface on essentially all exterior surface of the head and the roughened surface on essentially all exterior surface of the conduit.

90. A method according to either one of example implementation combination 85 or example implementation combination 86, wherein the roughened exposed surface extends over essentially all exterior surface of the modified form and the second treating comprises forming the smoothed exposed surface over essentially all of the roughened exposed surface.

91. A method according to any one of example implementation combinations 85-90, wherein the second treating comprises forming a coating of material over the at least a portion of the exposed roughened surface.

92. A method according to example implementation combination 91, wherein the forming a coating comprises dip coating material over the at least a portion of the roughened exposed surface.

93. A method according to example implementation combination 92, wherein the dip coating comprises:

dipping into a dip-coating formulation at least a portion of the head adjacent a proximal end of the internal passage with a mandrel extending through the internal passage and an extension portion of the mandrel extending out of the proximal end of the internal passage, whereby the extension portion of the mandrel is coated with the dip-coating formulation during the dipping; and

after the dipping, retracting the mandrel to retract the extension portion of the mandrel into the internal passage to coat at least a proximal portion of the internal passage with dip-coating formulation transferred from the extension portion of the mandrel.

94. A method according to example implementation combination 93, wherein the dip coating comprises multiple dip coat steps, wherein a first said dip coat step coats both a proximal side and a distal side of each said flanged portion of the head and second said dip coat step coats the proximal side but not the distal side of each said flanged portion of the head.

95. A method according to any one of example implementation combinations 91-94, wherein the material of the coating is the same as material of the exposed roughened surface.

96. A method according to any one of example implementation combinations 91-94, wherein the material of the coating is different than material of the exposed roughened surface.

97. A method according to either one of example implementation combination 95 or example implementation combination 96, any one of example implementation combinations 91-96, wherein the material of the coating and the material of the roughened surface are each a silicone material.

98. A method according to any one of example implementation combinations 92-97, wherein the dip coating comprises forming a layer of material over the at least a portion of the roughened exposed surface that is not thicker than 50 microns.

99. A method according to any one of example implementation combinations 75-98, wherein each said flanged portion of the head includes a concave undercut portion on a distal side of the said flanged portion and the roughened exterior surface includes essentially all exterior surface of the distal side.

100. A method for making a paranasal access implant device, wherein the paranasal implant device comprises:

a proximal end at a first longitudinal end of the device to be disposed in the lacrimal apparatus when the device is implanted and a distal end at a second longitudinal end of the device to be disposed in the paranasal sinus when the device is implanted;

a length longitudinally along the implant device between the proximal end and the distal end in a range of from 8 millimeters to 50 millimeters;

a conduit, located between the proximal end and distal end, to be disposed through a fistula between the lacrimal apparatus and the paranasal sinus when the device is implanted, the conduit comprising a distal portion to be disposed in the paranasal sinus when the implant device is implanted;

a head adjacent the proximal end and connected with the conduit, the head including at least one flanged portion to be disposed in the lacrimal apparatus when the device is implanted, the flanged portion having a distal side to be disposed toward tissue adjacent the fistula when the implant device is implanted and a proximal side opposite the distal side;

an internal passage through the head and the conduit, to provide a fluid communication path between the lacrimal apparatus and the paranasal sinus when the device is implanted, the internal passage having a width through at least a portion of the conduit in a range of from 0.5 millimeter to 2.5 millimeters;

wherein:

the flanged portion of the head has a concave undercut portion on the distal side, and the method comprises modifying a preliminary form of the implant device, the preliminary form including preliminary forms for the head, conduit and internal passage, with the preliminary form of the head including a preliminary form of the concave undercut portion, and the modifying comprises enlarging the head by adding a layer of added material to the proximal side and the distal side of the head, wherein the layer of added material is at least twice as thick on the proximal side of the head as on the concave undercut portion of the distal side of the head.

101. A method according to example implementation combination 100, wherein the layer of added material on the proximal side has a thickness in a range of from 10 microns to 500 microns.

102. A method according to either one of example implementation combination 100 or example implementation combination 101, wherein the layer of added material on the concave undercut portion of the distal side of the head has a thickness in a range of from 5 microns to 100 microns.

103. A method according to any one of example implementation combinations 100-102, wherein the layer of added material is at least five times as thick on the proximal side of the head as on the concave undercut portion of the distal side of the head.

104. A method according to any one of claims 100-103, wherein the modifying comprises:

first adding a first layer portion of added material to both the proximal side and the distal side of the head;

after the first adding, second adding a second layer portion of added material to the proximal side of the head and not to the distal side of the head.

105. A method according to example implementation combination 104, wherein the first adding includes a first dip coating step during which the proximal side and distal side of the head are dipped into a first dip-coating formulation and the second adding and the second adding includes a second dip coating step during which the proximal side but not the distal side of the head is dipped into a second dip-coating formulation.

106. A method according to example implementation combination 105, wherein the second dip-coating formulation comprises a higher concentration of added material precursor than the first dip-coating formulation.

107. A method according to any one of example implementation combinations 100-106, wherein exposed surfaces of the added layer on the proximal side and on the distal side of the head each has a smaller average roughness than exposed surfaces of each of the proximal side and the distal side of the preliminary form of the head prior to the modifying.

108. A method according to example implementation combination 107, wherein the exposed surfaces of the added layer on the proximal side and on the distal side of the head have an average roughness of not larger than 25 nanometers and the exposed surfaces of the proximal side and the distal side of the preliminary form of the head prior to the modifying have an average roughness of larger than 100 nanometers.

109. A method according to any one of Claims 75-108, wherein the implant device is according to any one of Claims 1-74.

110. A paranasal sinus access implant device according to any one of example implementation combinations 1-108, wherein the implant device comprises at least one of the following:
(i) the conduit comprises a first material having a first hardness and the head comprises a second material having a second hardness that is smaller than the first hardness;
(ii) the conduit comprises a distal portion to be disposed in the paranasal sinus when the device is implanted, the distal portion of the conduit comprising a structural portion of a first material having a first hardness and a skin portion supported by the structural portion, the skin portion including an a second material having a second hardness that is smaller than the first hardness;
(iii) the head comprises a structural portion of a first material having a first hardness and a skin portion supported by the structural portion, the skin portion including a second material having a second hardness that is smaller than the first hardness;
(iv) the head has an exposed surface of a second material having a hardness of not larger than Shore A 45 durometer;
(v) the head comprises an exposed surface of a second material, the exposed surface having an average roughness (Ra) of not larger than 200 nanometers, or even not larger than 50 nanometers;
(vi) the conduit comprises a distal portion to be disposed in the paranasal sinus when the device is implanted, the distal portion of the conduit comprising an exposed surface of a second material, the exposed surface having an average roughness (Ra) of not larger than 200 nanometers, or even not larger than 50 nanometers;
(vii) the head comprises an exposed surface of a second material comprising a wetting agent to impart hydrophilicity to the exposed surface;
(viii) the conduit comprises a distal portion to be disposed in the paranasal sinus when the device is implanted, the distal portion of the conduit comprising an exposed surface of a second material comprising a wetting agent to impart hydrophilicity to the exposed surface;
(ix) the head comprises an exposed surface of a second material comprising an antimicrobial agent;
(x) the conduit comprises a distal portion to be disposed in the paranasal sinus when the device is implanted, the distal portion of the conduit comprising an exposed surface of a second material comprising an antimicrobial agent;
(xi) the head comprises a distal side having a concave surface disposed toward the distal end of the device;
(xii) the internal passage has a surface of a second material comprising a lubricity agent;
(xiii) at least a portion of the conduit is of a radiopaque material;
(xiv) the internal passage has a surface geometry comprising rifling;
(xv) the conduit comprises a distal extension portion that is extendable and collapsible to lengthen and shorten a longitudinal length of a distal portion of the conduit disposed in the paranasal sinus when the device is implanted; and
(xvi) the implant device is packaged in sterile packaging in contact with a storage liquid.

111. An implant device according to example implementation combination 110, comprising one or more of (i)-(iii) and wherein the first material has a hardness in a range of from Shore A 50 durometer to Shore A 100 durometer.

112. An implant device according to example implementation combination 111, wherein the first material comprises a silicone material.

113. An implant device according to example implementation combination 112, wherein the silicone material comprises polydimethylsiloxane.

114. An implant device according to example implementation combination 113, wherein the first material comprises a polyurethane.

115. An implant device according to any one of example implementation combinations 2-5 wherein the first material has a hardness that is at least 20 Shore A durometer units larger than a hardness of the second material.

116. An implant device according to any one of example implementation combinations 110-115, comprising one or more of (i)-(x) and wherein the second material has a hardness in a range of from Shore A 5 durometer to Shore A 45 durometer.

117. An implant device according to example implementation combination 116, wherein the second material comprises a silicone material.

118. An implant device according to example implementation combination 116, wherein the second material comprises a silicone hydrogel.

119. An implant device according to any one of example implementation combinations 116-118, comprising one or both of items (ii) and (iii) and wherein the skin portion has a depth below the exposed surface in a range of from 10 microns to 200 microns.

120. An implant device according to any one of example implementation combinations 116-119, comprising one or both of (v) and (vi) and wherein the average roughness (Ra) is in a range of from 1 nanometer to 20 nanometers.

121. An implant device according to any one of example implementation combinations 116-120, comprising one or both of (vii) and (viii) and wherein the wetting agent is selected from the group consisting of polyvinylpyrrolidone, polyethylene glycol, hyaluronic acid and combinations thereof.

122. An implant device according to any one of example implementation combinations 116-121, comprising one or both of (ix) and (x) and wherein the antimicrobial agent comprises a member selected from the group consisting of silver, poly(ethylene oxide), polyethylene glycol and combinations thereof.

123. An implant device according to any one of example implementation combinations 116-122, comprising one or both of (ix) and (x) and wherein the antimicrobial agent comprises an antimicrobial peptide.

124. An implant device according to any one of example implementation combinations 116-123, comprising one or both if (ix) and (x) and wherein the second material comprises a polymeric material impregnated with the antimicrobial agent.

125. An implant device according to any one of example implementation combinations 110-124, comprising one or more of (i)-(iii), wherein a third material is disposed between the first material and the second material, the third material having a third hardness that is smaller than the first hardness and larger than the second hardness.

126. An implant device according to example implementation combination 125, wherein the third hardness is at least 10 Shore A durometer units smaller than the first hardness and the second hardness is at least 10 Shore A durometer units smaller than the third hardness.

127. An implant device according to either one of example implementation combinations 125 or claim 126, wherein the third harness is in a range of from Shore A 20 durometer to Shore A 50 durometer.

128. An implant device according to any one of example implementation combinations 125-127-, wherein the third material comprises a silicone material.

129. An implant device according to any one of example implementation combinations 110-128, comprising (xii) and wherein the lubricity agent is selected from the group consisting of a fluorosilicone, a smooth silicone film having an average roughness Ra of not larger than 50 nanometers, a poly (p-xylylene) and combinations thereof.

130. An implant device according to any one of example implementation combinations 110-129, comprising (xii) and wherein the lubricity agent is in the form of a coating on walls of the internal passage.

131. An implant device according to example implementation combination 130, wherein the coating has a thickness in a range of from 1 micron to 50 microns.

132. An implant device according to any one of example implementation combinations 110-131, comprising (xiii) and wherein the radiopaque material comprises a polymeric material mixed with a radiopaque additive.

133. An implant device according to example implementation combination 23, wherein the radiopaque additive is selected from the group consisting of barium sulfate, titanium metal, tantalum metal, gold metal, platinum metal, iodine, bismuth subcarbonate, bismuth trioxide, bismuth oxychloride, tungsten metal and combinations thereof.

134. An implant device according to either one of example implementation combination 132 or example implementation combination 133, wherein the radiopaque material comprises the radiopaque additive in an amount in a range of from 1 weight % to 90 weight %.

135. An implant device according to any one of example implementation combinations 110-134, comprising (xiii) and wherein at least a portion of the conduit has a radiodensity in a range of from 100 Hounsfield units to 900 Hounsfield units.

136. An implant device according to any one of example implementation combinations 110-135, comprising (xiii) and wherein the conduit has a radiopaque portion made of the radiopaque material, the radiopaque portion of the conduit extending for at least 5 millimeters of a longitudinal length of the conduit.

137. An implant device according to example implementation combination 136, wherein a proximal end of the radiopaque portion of the conduit is spaced at least 0.5 millimeter from the head.

138. An implant device according to example implementation combination 136, wherein the radiopaque portion extends over the entire longitudinal length of the conduit.

139. An implant device according to any one of example implementation combinations 110-138, comprising (xiii) and wherein the head does not contain a radiopaque additive.

140. An implant device according to any one of example implementation combinations 110-139, comprising (xiii) and wherein at least a portion of the head has a radiodensity of no larger than 50 Hounsfield units.

141. An implant device according to example implementation combination 140, wherein the entire head has a radiodensity of not larger than 75 Hounsfield units.

142. An implant device according to any one of example implementation combinations 110-141, wherein the head has a length dimension and a width dimension transverse to the length dimension with a ratio of the length dimension to the width dimension in a range of from 1.5 to 4.

143. An implant device according to any one of example implementation combinations 110-142, wherein the head has a length dimension and a width dimension transverse to the length dimension, the length dimension being in a range of from 3 millimeters to 8 millimeters.

144. An implant device according to any one of example implementation combinations 110-143, wherein an exterior of the conduit comprises an anchoring surface feature including protrusion areas and recess areas.

145. An implant device according to either one of example implementation combination 143 or example implementation combination 145, wherein the protrusion areas are on a longitudinal portion of the conduit having a proximal end that is disposed at least 3 millimeters distal of the head.

146. An implant device according to any one of example implementation combinations 110-145, wherein the length between the proximal end and the distal end of the device is in a range of from 8 millimeters to 30 millimeters.

147. An implant device according to any one of example implementation combinations 110-146, wherein the extension portion comprises a pleated structure.

148. A method of making a paranasal sinus access implant device according to any one of example implementation combinations 110-147 and 187-192, wherein the paranasal sinus access implant device comprises at least one said second material, the method comprising:

providing a preliminary form including a preliminary head structure and a preliminary conduit structure; and

forming at least one said second material supported by one or both of the preliminary head structure and the preliminary conduit structure.

149. A method according to example implementation combination 148, wherein the implant device comprises (i), the preliminary conduit structure of the preliminary form comprises the first material as recited in (i) and the forming at least one said second material comprises:

forming over at least a portion of the preliminary head structure a said second material as recited in (i) with the exposed surface as recited in (i).

150. A method according to either one of example implementation combination 148 or example implementation combination 40, wherein the implant device comprises (ii), the preliminary conduit structure comprises the first material as recited in (ii) and the forming at least one said second material comprises:

forming over at least a portion of said first material as recited in (ii) of the preliminary conduit structure a said second material as recited in (ii) with the exposed surface as recited in (ii).

151. A method according to any one of example implementation combinations 148-150, wherein the implant device comprises (iii) and the preliminary head structure of the preliminary form comprises the first material as recited in (iii), and the forming at least one said second material comprises:

forming over at least a portion of said first material as recited in (iii) of the preliminary head structure a said second material as recited (iii) with the exposed surface as recited in (iii).

152. A method according to any one of example implementation combinations 148-151, wherein the implant device comprises (iv) and the forming at least one said second material comprises:

forming over at least a portion of the preliminary head structure a said second material as recited in (iv) with the exposed surface recited in (iv).

153. A method according to any one of example implementation combinations 148-152, wherein the implant device comprises (v) and the forming at least one said second material comprises:

forming over at least a portion of the preliminary head structure a said second material as recited in (v) with the exposed surface as recited in (v).

154. A method according to any one of example implementation combinations 148-153, wherein the implant device comprises (vi) and the forming at least one said second material comprises:

forming over at least a portion of the preliminary conduit structure a said second material as recited in (vi) with the exposed surface as recited in (vi).

155. A method according to any one of example implementation combinations 148-154, wherein the implant device comprises (vii) and the forming at least one said second material comprises:

forming over at least a portion of the preliminary head structure a said second material as recited in (vii) with the exposed surface as recited in (vii).

156. A method according to any one of example implementation combinations 148-155, wherein the implant device comprises (viii) and the forming at least one said second material comprises:

forming over at least a portion of the preliminary conduit structure a said second material as recited in (viii) with the exposed surface as recited in (viii).

157. A method according to any one of example implementation combinations 148-156, wherein the implant device comprises (ix) and the forming at least one said second material comprises:

forming over at least a portion of the preliminary head structure a said second material as recited in (ix) with the exposed surface as recited in (ix).

158. A method according to any one of example implementation combinations 148-157, wherein the implant device comprises (x) and the forming at least one said second material comprises:

forming over at least a portion of the preliminary conduit structure a said second material as recited in (x) with the exposed surface as recited in (x).

159. A method according to any one of example implementation combinations 148-158, wherein paranasal sinus access implant device comprises (xii), the preliminary form comprises a preliminary internal passage through the preliminary conduit structure and the preliminary head structure and the method comprises:

forming over a wall of the preliminary internal passage a said second material as recited in (xii) with the exposed surface recited in (xii).

160. A method according to any one of example implementation combinations 148-159, wherein the providing a preliminary form comprises:

molding a polymeric composition in the shape of the preliminary form.

161. A method according to example implementation combination 150, wherein the molding comprises a molding technique selected from the group consisting of injection molding, compression molding and transfer molding.

162. A method according to any one of example implementation combinations 148-161, wherein the providing a preliminary form comprises:

extruding a first preliminary form and then molding additional features onto the first preliminary form to form a second preliminary form.

163. A method according to only one of example implementation combinations 160-162, wherein the providing a preliminary form comprises:

removing flash from a molded article resulting from the molding.

164. A method according to any one of example implementation combinations 148-163, wherein the forming at least one said second material comprises:

dip molding a said second material over at least a portion of said preliminary head structure.

165. A method according to any one of example implementation combinations 148-164, wherein the forming at least one said second material comprises:

dip molding a said second material over at least a portion of said preliminary conduit structure.

166. A method according to either one of example implementation combination 164 or example implementation combination 165, wherein the dip molding comprises:

applying at least a portion of the preliminary form with a precursor solution comprising at least one precursor for a said second material;

drying the applied precursor solution to leave the at least one precursor on the preliminary form; and

curing the at least one precursor to form a polymeric composition of a said second material.

167. A method according to example implementation combination 166, wherein the polymeric composition is a thermoset composition, and the curing comprises heating the at least one precursor on the preliminary form.

168. A method according to any one of example implementation combinations 48-167, wherein the forming at least one said second material comprises:

forming an intermediate material over at least a portion of one or both of the preliminary head structure and a preliminary conduit structure; and

after the forming an intermediate material, forming the second material over at least a portion of the preliminary material.

169. A method according to any one of example implementation combinations 148-168, wherein the forming at least one said second material comprises:

forming a preliminary material over at least a portion of one or both of the preliminary head structure and a preliminary conduit structure; and

after the forming a preliminary material, modifying the preliminary material to form a said second material.

170. A method according to example implementation combination 169, wherein the modifying the preliminary material comprises adding to the preliminary material an additive material.

171. A method according to example implementation combination 170, wherein the additive material is selected from the group consisting of a said wetting agent, a said antimicrobial agent and combinations thereof.

172. A method of treating a condition of a paranasal sinus of a patient having the implant device of any one of example implementation combinations 110-147 and 187-192 implanted to fluidly connect a lacrimal apparatus of the patient to a paranasal sinus of the patient, the method comprising the administering a treatment composition to the patient to be delivered to the paranasal sinus through the internal passage of the implant device.

173. A product, comprising:

a paranasal sinus access implant device;

sterile storage liquid in contact with the implant device;

sterile packaging, wherein the implant device and the storage liquid are disposed within the sterile packaging.

174. A product according to example implementation combination 173, wherein the implant device comprises:

a proximal end at a first longitudinal end of the device to be disposed in the lacrimal apparatus when the device is implanted and a distal end at a second longitudinal end of the device to be disposed in the paranasal sinus when the device is implanted;

a length longitudinally along the implant device between the proximal end and the distal end in a range of from 2 millimeters to 50 millimeters.

a conduit, located between the proximal end and distal end, to be disposed through a fistula between the lacrimal apparatus and the paranasal sinus when the device is implanted;

a head located proximal of the conduit, to be disposed in the lacrimal apparatus when the device is implanted;

an internal passage through the head and the conduit, to provide a fluid communication path between the lacrimal apparatus and the paranasal sinus when the device is implanted, the internal passage having a width through at least a portion of the conduit in a range of from 0.25 millimeter to 5 millimeters.

175. A product according to either one of example implementation combination 173 and example implementation combination 174, wherein all exterior surfaces of the implant device are in contact with the storage liquid.

176. A product according to any one of example implementation combinations 173-175, wherein all surfaces of the implant device are in contact with the storage liquid.

177. A product according to any one of example implementation combinations 173-176, wherein the implant device is disposed within the sterile packaging in a reservoir of the storage liquid.

178. A product according to any one of example implementation combinations 173-177, wherein the storage liquid comprises an aqueous liquid.

179. A product according to any one of example implementation combinations 173-178, wherein the storage liquid comprises a buffer solution.

180. A product according to any one of example implementation combinations 173-179, wherein the storage liquid comprises a wetting agent.

181. A product according to example implementation combination 180, wherein the wetting agent comprises hyaluronic acid.

182. A product according to any one of example implementation combinations 173-181, wherein the product comprises a plurality of said implant devices and the sterile packaging comprises a plurality of sealed compartments each having disposed therein a said implant device in contact with a said storage liquid.

183. A product according to any one of example implementation combinations 173-182, wherein the implant device is according to any one of example implementation combinations 1-38 and 78-83.

184. A product according to example implementation combination 183, wherein the implant device comprises (xvi).

185. A method for implanting a paranasal sinus access implant device to fluidly connect a lacrimal apparatus and a paranasal sinus, the method comprising:

from the product of any one of example implementation combinations 173-184, removing the implant device from the sterile packaging; and

implanting the implant device with a proximal end disposed in the lacrimal apparatus and a distal end disposed in the paranasal sinus to fluidly connect the lacrimal apparatus and the paranasal sinus through an internal passage of the implant device.

186. A method according to example implementation combination 185, wherein after the removing and at the commencement of the implanting, at least a portion of the implant device is covered with at least a residual portion of the storage liquid.

187. An implant device according to any one of example implementation combinations 110-147, comprising a said exposed surface having has an area of at least 1 square millimeter.

188. An implant device according to any one of example implementation combinations 110-147 and 187 comprising a said exposed surface on an exterior of the distal portion of the conduit, which distal portion of the conduit is to be disposed in the paranasal sinus when the implant device is implanted.

189. An implant device according to example implementation combination 188, wherein a said exposed surface extends for at least 2 millimeters along a longitudinal length of the conduit adjacent to the distal end.

190. An implant device according to either one of example implementation combination 186 and example implementation combination 189, wherein a said exposed surface extends entirely around an exterior circumference of the distal portion of the conduit.

191. An implant device according to any one of example implementations 110-148 and 187-190, wherein the Ra is as determined by optical non-contact profilometry or by laser profilometry.

192. An implant device according to any one of example implementations 110-148 and 187-191, wherein the head comprises flanged portions having a refractive index across the thickness of the flanged portions of not larger than 1.5.

193. An implant device according to any one of example implementation combinations 110-148 and 187-192, wherein the implant device is implanted in a human and fluidly connects a location in the lacrimal apparatus with a paranasal sinus.

The foregoing discussion of the invention and different aspects and different example implementation combinations thereof has been presented for purposes of illustration and description. The foregoing is not intended to limit the invention to only the form or forms specifically disclosed herein. Consequently, variations and modifications commensurate with the above teachings, and the skill or knowledge of the relevant art, are within the scope of the present invention. The embodiments described hereinabove are further intended to explain best modes known for practicing the invention and to enable others skilled in the art to utilize the invention in such, or other, embodiments and with various modifications required by the particular applications or uses of the present invention. It is intended that the appended claims be construed to include alternative embodiments to the extent permitted by the prior art. Although the description of the invention has included description of one or more possible implementations and certain variations and modifications, other variations and modifications are within the scope of the invention, e.g., as may be within the skill and knowledge of those in the art after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter. Furthermore, any feature described or claimed with respect to any disclosed implementation may be combined in any combination with one or more of any other features of any other implementation or implementations, to the extent that the features are not necessarily technically incompatible, and all such combinations are within the scope of the present invention.

The terms “comprising”, “containing”, “including” and “having”, and grammatical variations of those terms, are intended to be inclusive and nonlimiting in that the use of such terms indicates the presence of some condition or feature, but not to the exclusion of the presence also of any other condition or feature. The use of the terms “comprising”, “containing”, “including” and “having”, and grammatical variations of those terms in referring to the presence of one or more components, subcomponents or materials, also include and is intended to disclose the more specific embodiments in which the term “comprising”, “containing”, “including” or “having” (or the variation of such term) as the case may be, is replaced by any of the narrower terms “consisting essentially of” or “consisting of” or “consisting of only” (or the appropriate grammatical variation of such narrower terms). For example, a statement that some thing “comprises” a stated element or elements is also intended to include and disclose the more specific narrower embodiments of the thing “consisting essentially of” the stated element or elements, and the thing “consisting of” the stated element or elements. Examples of various features have been provided for purposes of illustration, and the terms “example”, “for example” and the like indicate illustrative examples that are not limiting and are not to be construed or interpreted as limiting a feature or features to any particular example. The term “at least” followed by a number (e.g., “at least one”) means that number or more than that number. The term “at least a portion” means all or a portion that is less than all. The term “at least a part” means all or a part that is less than all.

The terms “essentially all” or “substantially all” in relation to a surface or material means an amount of the surface or material that is a practical functional equivalent of all of the surface or material, and in any event includes a quantity of such surface or material that is 90 percent or more of all of such surface or material.

The features in the drawings are shown for illustration purposes and to generally show relative positioning and interaction, and the features shown are not necessarily to scale.

1. A paranasal sinus access implant device useful for implantation in a human to fluidly connect a lacrimal apparatus to a paranasal sinus through a fistula formed between the lacrimal apparatus and the paranasal sinus, the implant device comprising:
a proximal end at a first longitudinal end of the device to be disposed in the lacrimal apparatus when the device is implanted and a distal end at a second longitudinal end of the device to be disposed in the paranasal sinus when the device is implanted;
a length longitudinally along the implant device between the proximal end and the distal end in a range of from 8 millimeters to 50 millimeters;
a conduit, located between the proximal end and distal end, to be disposed through a fistula between the lacrimal apparatus and the paranasal sinus when the device is implanted, the conduit comprising a distal portion to be disposed in the paranasal sinus when the implant device is implanted;
a head adjacent the proximal end and connected with the conduit, the head including a flanged portion to be disposed in the lacrimal apparatus when the device is implanted; and
an internal passage through the head and the conduit, to provide a fluid communication path between the lacrimal apparatus and the paranasal sinus when the device is implanted, the internal passage having a width through at least a portion of the conduit in a range of from 0.5 millimeter to 2.5 millimeters; and
wherein the head has a perimeter transverse to a longitudinal axis of the implant device and the flanged portion of the head extends 360 degrees around the axis and includes a concave undercut portion on a distal side of the flanged portion, with the concave undercut extending 360 degrees around the axis within the perimeter of the head.

2. An implant device according to claim 1, wherein the concave undercut has an average roughness of not larger than 50 nanometers.

3. An implant device according to claim 1, wherein the perimeter of the head is circular and the circle of the perimeter of the head has a diameter in a range of from 2.75 millimeters to 6 millimeters.

4. An implant device according to claim 1, wherein the concave undercut has a uniform cross-section in a plane including the axis at all radial locations about the axis.

5. An implant device according to claim 1, wherein the concave undercut has a radius of curvature in a range of from 0.1 millimeter to 0.5 millimeter.

6. An implant device according to claim 1, wherein the concave undercut has a depth relative to a distal lip of the flanged portion in a range of from 0.1 millimeter to 0.6 millimeter.

7. An implant device according to claim 1, wherein the head has a depth dimension in a direction from the proximal end to the distal end of the implant device in a range of from 0.4 millimeter to 1.25 millimeters.

8. An implant device according to claim 1, wherein the internal passage includes a proximal port transition portion including a tapering cross-section that tapers in a direction from the proximal end toward the distal end of the implant device.

9. An implant device according to claim 8, wherein;
the proximal port transition portion is in the head;
at all locations of the proximal port transition portion in a longitudinal direction from the proximal end to the distal end of the implant device, the proximal port transition portion has a circular cross-section normal to a longitudinal axis of the implant device;
the diameter of the circular cross-section of the internal passage tapers by at least 0.1 millimeter from an initial diameter at a proximal end of the proximal port transition portion in a range of from 0.75 millimeter to 3.5 millimeters to a tapered diameter at a distal end of the proximal port transition portion in a range of from 0.5 millimeter to 2.5 millimeters; and
the proximal port transition portion has a length from a proximal end to a distal end of the proximal port transition portion in a range of from 0.4 millimeter to 1.5 millimeters.

10. An implant device according to claim 1, wherein the internal passage includes a distal port transition portion in a distal portion of the conduit, the distal port transition portion having a tapering cross-section that tapers in a direction from the distal end toward the proximal end of the implant device.

11. An implant device according to claim 10, wherein:
at all locations of the distal port transition portion in a longitudinal direction from the proximal end to the distal end of the implant device, the distal port transition portion has a circular cross-section normal to a longitudinal axis of the implant device;
the diameter of the circular cross-section of the internal passage in the distal port transition portion tapers by an amount in a range of from 0.25 millimeter to 2 millimeter from a distal end of the distal port transition portion to a proximal end of the distal port transition portion, and the proximal end of the distal transition portion has a circular cross-section with a diameter in a range of from 0.5 millimeter to 2.5 millimeter: and
the distal port transition portion has a length from a proximal end to a distal end of the distal port transition portion in a range of from 0.25 millimeter to 2 millimeters.

12. An implant device according to claim 1, comprising an exposed surface of the interior passage having an average roughness (Ra) of not larger than 50 nanometers and the exposed surface of the interior passage includes essentially all surface exposed in the internal passage.

13. An implant device according to claim 12, wherein the internal passage is free of mold flash.

14. An implant device according to claim 1, comprising a first exposed surface having a first average roughness of not larger than 50 nanometers and a second exposed surface having a second average roughness of at least 100 nanometers, and wherein the second exposed surface includes an exposed exterior surface of the conduit; and
the first exposed surface includes an exposed exterior surface of the head.

15-16. (canceled)

17. An implant device according to claim 1, comprising an exposed surface over essentially all exterior of the implant device having an average roughness of not larger than 50 nanometers.

18. An implant device according to claim 1, wherein:
an exterior of the conduit comprises an anchoring surface feature including protrusion areas and recess areas, with the protrusion areas including protrusion occurrences in the form of circumferential ridges each having a circular perimeter normal to the longitudinal axis of the implant device and spaced along the conduit and separated by the recess areas, wherein the anchoring surface feature includes at least 5 of the circumferential ridges, wherein the anchor surface feature includes at least 5 of the circumferential ridges;
the circular perimeter of each said circumferential ridge has a diameter of in a range of from 2.3 millimeters to 2.6 millimeters.
the circumferential ridges have a height relative to the recess areas in a range of from 0.25 millimeter to 0.5 millimeter;
the circumferential ridges have a center-to-center spacing in a range of from 1.2 millimeter to 1.9 millimeter;
a nearest said circumferential ridge to the proximal end of the implant device is spaced a distance in a range of from 7 to 11 millimeters from the proximal end of the implant device;
between a proximal end of the conduit and a nearest said circumferential ridge to the proximal end of the implant device, the conduit has a uniform circular perimeter normal to the longitudinal axis of the implant device having a first diameter in a range of from 1.9 millimeters to 2.1 millimeters; and
at the recess areas the conduit has a circular perimeter normal to the longitudinal axis of the implant device having a second diameter that is smaller than the first diameter and that is in a range of from 1.6 millimeters to 1.9 millimeters.

19. An implant device according to claim 18, comprising at least one side port through a wall of the conduit into the internal passage, wherein the side port is located between two adjacent ones of the circumferential ridges and the side port is encircled by a raised ridge on the exterior of the conduit between the two said adjacent circumferential ridges, the raised circular ridge that is raised relative to the recess area between the two said adjacent circumferential ridges; and
the raised circular ridge is raised relative to the adjacent recessed area by no more than 0.2 millimeter.

20. (canceled)

21. A method for making a paranasal access implant device constructed of polymeric material, wherein the paranasal access implant device is according to claim 1 the method comprises:
preparing a preliminary form of the implant device including preliminary forms of the head, conduit and internal passage; and
modifying the preliminary form, wherein the modifying comprises treating an exposed surface on an exterior of the preliminary form to prepare a roughened exposed surface having a higher average roughness than the exposed surface prior to the treating.

22-24. (canceled)

25. A method for making a paranasal access implant device, wherein the paranasal implant device comprises:
a proximal end at a first longitudinal end of the device to be disposed in the lacrimal apparatus when the device is implanted and a distal end at a second longitudinal end of the device to be disposed in the paranasal sinus when the device is implanted;
a length longitudinally along the implant device between the proximal end and the distal end in a range of from 8 millimeters to 50 millimeters;
a conduit, located between the proximal end and distal end, to be disposed through a fistula between the lacrimal apparatus and the paranasal sinus when the device is implanted, the conduit comprising a distal portion to be disposed in the paranasal sinus when the implant device is implanted;
a head adjacent the proximal end and connected with the conduit, the head including at least one flanged portion to be disposed in the lacrimal apparatus when the device is implanted, the flanged portion having a distal side to be disposed toward tissue adjacent the fistula when the implant device is implanted and a proximal side opposite the distal side;
an internal passage through the head and the conduit, to provide a fluid communication path between the lacrimal apparatus and the paranasal sinus when the device is implanted, the internal passage having a width through at least a portion of the conduit in a range of from 0.5 millimeter to 2.5 millimeters;
wherein:
the flanged portion of the head has a concave undercut portion on the distal side, and the method comprises modifying a preliminary form of the implant device, the preliminary form including preliminary forms for the head, conduit and internal passage, with the preliminary form of the head including a preliminary form of the concave undercut portion, and the modifying comprises enlarging the head by adding a layer of added material to the proximal side and the distal side of the head, wherein the layer of added material is at least twice as thick on the proximal side of the head as on the concave undercut portion of the distal side of the head.

26-29. (canceled)

30. A method according to claim 1, wherein:
the concave undercut has an average roughness of not larger than 50 nanometers;
the perimeter of the head is circular and the circle of the perimeter of the head has a diameter in a range of from 2.75 millimeters to 6 millimeters;
the concave undercut has a uniform cross-section in a plane including the axis at all radial locations about the axis;
the concave undercut has a radius of curvature in a range of from 0.1 millimeter to 0.5 millimeter;
the concave undercut has a depth relative to a distal lip of the flanged portion in a range of from 0.1 millimeter to 0.6 millimeter; and
the head has a depth dimension in a direction from the proximal end to the distal end of the implant device in a range of from 0.4 millimeter to 1.25 millimeters.
